(19) United States
(12) Patent Application Publication
(10) Pub. No.: US 2013/0283404 A1
(43) Pub. Date: Oct. 24, 2013

(54) EPIGENETICS IN AUTOIMMUNITY

(71) Applicants: Bruce C. Richardson, Ann Arbor, MI (US); Anura Hewagama, Ann Arbor, MI (US); Gabriela Gorelik, Ann Arbor, MI (US)

(72) Inventors: Bruce C. Richardson, Ann Arbor, MI (US); Anura Hewagama, Ann Arbor, MI (US); Gabriela Gorelik, Ann Arbor, MI (US)

(73) Assignee: The Regents of the University of Michigan, Ann Arbor, MI (US)

(21) Appl. No.: 13/828,388

(22) Filed: Mar. 14, 2013

Related U.S. Application Data
(60) Provisional application No. 61/618,160, filed on Mar. 30, 2012.

(51) Int. Cl. C12Q 1/68

(52) U.S. Cl. CPC C12Q 1/6883
USPC 800/14; 435/6.11

(57) ABSTRACT

Provided herein are compositions and methods for the diagnosis, monitoring, treatment, and/or prevention of autoimmune disease (e.g., lupus) based on a causal connection with various epigenetic markers (e.g., chromosome demethylation, overexpression of lupus markers, nitration of PKCδ in response to oxidative stress, etc.).

### CROSS REFERENCE TO RELATED APPLICATIONS

This application claims priority to U.S. Provisional Patent Application Ser. No. 61/618,160, filed Mar. 30, 2012, hereby incorporated by reference in its entirety.

### STATEMENT REGARDING FEDERALLY SPONSORED RESEARCH OR DEVELOPMENT

This invention was made with government support under AR042525 awarded by the National Institutes of Health. The government has certain rights in the invention.

### FIELD

Provided herein are compositions and methods for the diagnosis, monitoring, treatment, and/or prevention of autoimmune disease (e.g., lupus) based on a causal connection with various epigenetic markers (e.g., chromosome demethylation, overexpression of lupus markers, nitration of PKCδ in response to oxidative stress, etc.).

### BACKGROUND

Systemic lupus erythematosus (SLE or “lupus”) is an incompletely understood chronic, relapsing autoimmune disease. Symptoms vary from person to person, and may come and go. Almost all lupus sufferers experience joint pain and swelling, and some will develop arthritis. Frequently affected joints are the fingers, hands, wrists, and knees. Currently used lupus treatments are directed to controlling symptoms.

### SUMMARY

Provided herein are compositions and methods for the diagnosis, monitoring, treatment, and/or prevention of autoimmune disease (e.g., lupus) based on a causal connection with various epigenetic markers (e.g., chromosome demethylation, overexpression of lupus markers, nitration of PKCδ in response to oxidative stress, etc.). In some embodiments, compositions and methods are provided for the diagnosis, monitoring, treatment, and/or prevention of autoimmune disease (e.g., lupus). In particular, a causal connection between epigenetic modifications and lupus is described, and compositions and methods for the diagnosis, treatment, and/or prevention of lupus based thereon are provided. In certain embodiments, epigenetic markers comprise, for example, nitration of PKCδ; X-chromosome demethylation; overexpression of CD40LG, CXCR3, OGT, miR-98, let-7f-2, miR 188 3p, miR-421, miR-503; etc. In embodiments described herein, epigenetic markers of lupus are employed to: diagnose a subject with lupus or an increased risk of developing lupus, monitor the progression of (diagnosed) lupus, and/or monitor the effectiveness of therapies. In other embodiments, epigenetic markers of lupus are used to screen for therapies (e.g., therapeutics) useful in the treatment of, slowing the progression of, delaying/preventing the onset of, and/or reducing the risk of developing lupus. In certain embodiments, epigenetic markers of lupus provide targets for therapeutics for the treatment and/or prevention of lupus. In some embodiments, reagents for the detection, monitoring, or treatment of epigenetic markers of lupus are provided. In other embodiments, kits and reagents are provided for performing the above-mentioned methods (e.g., detecting, monitoring, screening, treating, etc.).

In some embodiments, the present invention provides methods of treating or preventing lupus in a subject by inhibiting and/or preventing modifications (e.g., nitration, oxidation, demethylation, etc.) to lupus-related markers. In some embodiments, the present invention provides a method of treating or preventing lupus in a subject by reversing and/or preventing oxidation-related modifications of PKCδ in the subject. In some embodiments, the oxidation-related modifications of PKCδ are reversed and/or prevented by reducing oxidative stress. In some embodiments, the lupus-related modifications comprise nitration of PKCδ. In some embodiments, the oxidation-related modifications of PKCδ result in differential phosphorylation of PKCδ, decreased phosphorylation of T⁵⁰⁵ of PKCδ, and/or decreased ERK signaling in lupus T cells. In some embodiments, antioxidants or other therapeutics are administered. In some embodiments, the present invention provides a method of treating or preventing lupus in a subject by reversing and/or preventing X-chromosome (e.g., inactive X (Xᵢ)) demethylation (e.g., in CD4+ cells). In some embodiments, overexpression of X-chromosome-linked lupus markers (e.g., CD40LG, CXCR3, OGT, miR-98, let-7f-2, miR 188 3p, miR-421, miR-503; etc.) is inhibited.

In some embodiments, the present invention provides a method of diagnosing lupus, assessing a subject's risk for developing lupus, and/or determining lupus disease activity in a subject comprising detecting and/or measuring one or more epigenetic markers of lupus. In some embodiments, the present invention provides a method of diagnosing lupus, assessing a subject's risk for developing lupus, and/or determining lupus disease activity in a subject comprising detecting oxidation-related modifications of PKCδ in the subject. In some embodiments, detecting oxidation-related modifications of PKCδ comprises determining the level of oxidation-related modifications of PKCδ in the subject. In some embodiments, a level of oxidation-related modifications of PKCδ above a control or threshold level is indicative of lupus, a risk of developing lupus, and/or active lupus in the subject. In some embodiments, oxidation-related modifications of PKCδ comprise nitration of PKCδ. In some embodiments, the present invention provides a method of diagnosing lupus, assessing a subject's risk for developing lupus, and/or determining lupus disease activity in a subject comprising detecting and/or quantitating X-chromosome (e.g., inactive X) demethylation (e.g., in CD4+ cells). In some embodiments, expression (e.g., overexpression) of X-chromosome-linked lupus markers (e.g., CD40LG, CXCR3, OGT, miR-98, let-7f-2, miR 188 3p, miR-421, miR-503; etc.) is detected and/or quantitated.

In some embodiments, diagnosing lupus, assessing a subject's risk for developing lupus, and/or determining lupus disease activity in a subject comprises the steps of: (a) obtaining a biological sample from the subject; (b) detecting and/or measuring one or more epigenetic markers of lupus in the sample; and (c) comparing the level epigenetic markers to a control or threshold level. In certain embodiments, epigenetic markers comprise oxidation-related modifications of PKCδ (e.g., in the T cells (e.g., CD4+ T cells) of the subject). In some embodiments, epigenetic markers comprise nitration of PKCδ. In some embodiments, epigenetic markers comprise demethylation of the (inactive) X-chromosome. In some embodiments, epigenetic markers comprise overexpression of X-chromosome-linked lupus markers (e.g., CD40LG, CXCR3, OGT, miR-98, let-7f-2, miR 188 3p, miR-421, miR-503. In some embodiments, methods further comprise isolating CD4+ T cells from the biological sample. In some embodiments, methods further comprise isolating PKCδ, X-chromosomes, CD40LG, CXCR3, OGT, miR-98, let-7f-2, miR 188 3p, miR-421, and/or miR-503 from the sample. In some embodiments, detecting and/or quantitating epigenetic markers (e.g., oxidation-related modifications of PKCδ, demethylation of X-chromosomes, overexpression of lupus markers, etc.) comprises in vitro analysis (e.g., antibody detection).

In some embodiments, the present invention provides methods of treating and/or preventing lupus comprising: (a) testing a subject for levels of epigenetic markers (e.g., oxidation-related modifications of PKCδ, demethylation of X-chromosomes, overexpression of lupus markers); (b) treating the subject with a lupus therapy (e.g., therapeutic, to reduce oxidative stress in the subject, etc.); and (c) re-testing the subject for levels of epigenetic markers. In some embodiments, epigenetic markers appear in the T cells (e.g., CD4+ T cells) of the subject. In some embodiments, the present invention provides methods monitoring treatment and/or prevention of lupus comprising: (a) treating a subject with an anti-lupus therapy (e.g., lupus therapeutic, to reduce oxidative stress in a subject, etc.); and (b) assessing the effect of the therapy on epigenetic markers of lupus (e.g., presence or levels). In some embodiments methods further comprise developing a treatment strategy based on the presence and/or levels of epigenetic lupus markers (e.g., in the T cells (e.g., CD4+ T cells) of the subject).

In some embodiments, diagnostic tests are provided for characterizing an individual's risk of developing or having an autoimmune disease (e.g., lupus). The present tests are useful for identifying those individuals who are in need of autoimmune (e.g., lupus) therapies as well as those individuals who require no therapies targeted at preventing such diseases. In certain embodiments, diagnostic tests are based on epigenetic markers of lupus (e.g., significantly greater levels of oxidation-modified PKCδ levels (e.g., in CD4+ T cells), X-chromosome demethylation, overexpression of lupus markers). Thus, the present diagnostic tests, which involve, for example, assessing levels of certain oxidation modification in PKCδ (e.g., types of modifications (e.g., nitration), modifications are certain positions, rate of modifications, etc.), detecting or quantifying X-chromosome demethylation, detecting or measuring expression of lupus markers (e.g., CD40LG, CXCR3, OGT, miR-98, let-7f-2, miR 188 3p, miR-421, miR-503) in a biological sample (e.g., blood sample) or derivative thereof (e.g., isolated CD4+ cells) from a test subject, provide additive predictive value beyond that seen with clinical and diagnostic risk factors currently employed by physicians.

In one aspect, the diagnostic tests provided herein comprise determining the level of oxidation-related modifications of PKCδ in a biological sample obtained from the individual or test subject. In some embodiments, the biological sample is blood or a derivative thereof (e.g., containing CD4+ T cells), including but not limited to, leukocytes, neutrophils, monocytes, serum, or plasma. The level of oxidation-related modifications of PKCδ in the biological sample from the test subject is then compared to a predetermined value (e.g., derived from measurements in comparable biological samples obtained from the general population or a select population of human subjects). Such comparison characterizes the test subject's risk of developing or having autoimmune disease (e.g., lupus). For example, test subjects whose blood levels of oxidation-related modifications of PKCδ are higher than the predetermined value are at greater risk of developing or having autoimmune disease (e.g., lupus) than individuals whose blood oxidation-related modifications of PKCδ are at or lower than the predetermined value. Moreover, the extent of the difference between the test subject's level of oxidation-related modifications of PKCδ and predetermined value is also useful for characterizing the extent of the risk and thereby, determining which individuals would most greatly benefit from certain therapies.

For those individuals who already exhibit symptoms of autoimmune disease (e.g., lupus) or have received a diagnosis based on a different diagnostic procedure, diagnostic tests provided herein are useful for assessing the activity or severity of the disease (e.g., lupus), and/or assessing the effectiveness of treatment. Thus, the present invention also provides a method for monitoring over time the status of autoimmune disease (e.g., lupus) in a subject. For example, methods comprise determining the levels of one or more of the present risk factors, including level of oxidative-related modifications of PKCδ, specific oxidative modification of PKCδ, PKCδ nitration, PKCδ phosphorylation, X-chromosome demethylation, lupus marker overexpression (e.g., CD40LG, CXCR3, OGT, miR-98, let-7f-2, miR 188 3p, miR-421, miR-503), and combinations thereof, in a biological sample taken from the subject at an initial time and in a corresponding biological sample taken from the subject at a subsequent time. An increase in the levels of the present risk factors from the biological sample taken at the subsequent time as compared to the initial time indicates that a subject's risk of developing an autoimmune disease, activity of autoimmune disease, and/or severity of autoimmune disease has increased. A decrease in the levels of the present risk factors from the biological sample taken at the subsequent time as compared to the initial time indicates that that the subject's risk of developing an autoimmune disease, activity of autoimmune disease, and/or severity of autoimmune disease has decreased.

In another aspect, the present invention provides a method for evaluating therapy in a subject suspected of having or having autoimmune disease (e.g., lupus). The method comprises determining the levels of one or more of the present risk factors, including level of oxidative-related modifications of PKCδ, specific oxidative modification of PKCδ, PKCδ nitration, PKCδ phosphorylation, X-chromosome demethylation, lupus marker overexpression (e.g., CD40LG, CXCR3, OGT, miR-98, let-7f-2, miR 188 3p, miR-421, miR-503), and combinations thereof, in a biological sample taken from the subject prior to therapy and a corresponding biological sample taken from the subject during or following therapy. A decrease in the level of the selected risk factors in the sample taken after or during therapy as compared to the level of the selected risk factors in the sample taken before therapy is indicative of a positive effect of the therapy on autoimmune disease (e.g., lupus) in the treated subject.

In some embodiments, the present invention provides methods of diagnosing lupus, assessing a subject's risk for developing lupus, and/or determining lupus disease activity in a subject comprising detecting one or more epigenetic markers of lupus. In some embodiments, detecting one or more epigenetic markers of lupus comprise oxidation-related modifications of PKCδ in the subject. In some embodiments, the oxidation-related modifications comprise nitration of PKC6. In some embodiments, detecting oxidation-related modifications of PKCδ comprises determining the level of oxidation-related modifications of PKCδ in the subject. In some embodiments, detecting oxidation-related modifications of PKCδ further comprises comparing the level of oxidation-related modifications of PKCδ to a control or threshold level that is indicative of lupus, a risk of developing lupus, and/or active lupus in the subject. In some embodiments, one or more epigenetic markers of lupus comprise X-chromosome demethylation. In some embodiments, the one or more epigenetic markers of lupus comprises overexpression of one or more of CD40LG, CXCR3, OGT, miR-98, let-7f-2, miR 188 3p, miR-421, and miR-503. In some embodiments, detecting one or more epigenetic markers of lupus comprises in vitro analysis. In some embodiments, detecting one or more epigenetic markers of lupus comprises antibody detection.

In some embodiments, the present invention provides methods of monitoring treatment of lupus comprising: (a) detecting one or more epigenetic markers of lupus; (b) administering a treatment for lupus to the subject; (c) repeating the detection of one or more epigenetic markers of lupus; (d) comparing the epigenetic markers detected in steps (a) and (c), wherein a reduction in one or more of the epigenetic markers of lupus indicates benefit of the treatment. In some embodiments, treating comprises reducing the oxidative stress in the subject. In some embodiments, treating comprises administering a lupus therapeutic. In some embodiments, one or more epigenetic markers of lupus comprise oxidation-related modifications of PKCδ in the subject. In some embodiments, the oxidation-related modifications comprise nitration of PKCδ. In some embodiments, the reduction in one or more of the epigenetic markers of lupus comprises a reduction in oxidation-related modifications of PKCδ. In some embodiments, the one or more epigenetic markers of lupus comprise X-chromosome demethylation. In some embodiments, the one or more epigenetic markers of lupus comprises overexpression of one or more of CD40LG, CXCR3, OGT, miR-98, let-7f-2, miR 188 3p, miR-421, and miR-503.

In some embodiments, the present invention provides non-human transgenic mammal exhibiting decreased expression and/or activity of PKCδ. In some embodiments, the mammal is a mouse. In some embodiments, decreased PKCδ expression and/or activity, when present, is limited to CD4+ cells. In some embodiments, PKCδ inactivation is only expressed in the presence of an inducer. In some embodiments, in the absence of PKCδ the mammal has lupus or a lupus-like condition. In some embodiments, the inducer comprises Doxycycline.

### DESCRIPTION OF THE DRAWINGS

FIG. 1 shows PDK-1 is not affected in lupus T cells. CD4+ T cells from healthy donors (control) or from lupus patients were stimulated or not with PMA for 15 min. 20 μg of whole cell lysates were subjected to SDS-PAGE fractionation, transferred to nitrocellulose membranes and probed with a polyclonal antibody against PKC δ p-T505. Membranes were stripped and reprobed with anti-phospho-PDK1. PKCδ and PDK-1 were used as controls for the corresponding total protein expression. Panel A shows a representative blot comparing phosphorylation of PDK1 in a lupus patient with a normal donor. Panel B represents the mean±S.E of four similar experiments performed in different cell preparations from normal and SLE patients.

FIG. 2 shows peroxynitrite decreases PKCδ T⁵⁰⁵ phosphorylation while increasing protein nitration in T cells. CD4+ T cells from healthy donors were untreated or treated with 20-80 μM peroxynitrite for 15 min and then PMA stimulated as indicated. Whole cell proteins were obtained and subjected to Western blot analysis. (A) Representative immunoblot showing phosphorylation at the activation loops of PKCδ, PKCθ and PKCα using antibodies specific to the phosphorylated PKC isoforms as indicated. (B) Quantitative analysis of PKCδ T505 from 4 healthy donors in four different experiments (mean±S.E.M). Values were normalized to total PKCδ. PMA-stimulated CD4+ T cells but without ONOO— treatment (solid bar) were considered as 100%. (C) Representative experiment using anti 3-nitrotyrosine antibody to probe PMA stimulated T cell lysates. D. Relative quantitation of 3-nitrotyrosine proteins from four similar experiments (mean±S.E.M.). Untreated cells and PMA-unstimulated cells (□) were used as controls.

FIG. 3 shows Differential PKCδ phosphorylation induced by peroxynitrite. (A) Representative experiment showing the effect of different ONOO⁻ concentrations on PKCδ phosphorylation. CD4+ T cells were isolated from healthy controls and treated with ONOO⁻ at the concentrations specified. Following treatment the cells were stimulated with 50 ng/ml PMA for 15 min and PMA-stimulated peroxynitrite-untreated cells were used as control. Protein lysates were then subjected to electrophoresis, transferred to nitrocellulose and the membranes probed with anti-p-T⁵⁰⁵-PKCδ. The blot was then stripped and reprobed with anti-p-Y³¹¹-PKC δ. NaOH was used as vehicle control and added to the culture for 15 min before PMA stimulation at the same final concentration as in the ONOO— solution. (B) Differential dose-response curves of p-PKCδ following ONOO— treatment. The graph represents the mean±SEM of p-PKCδ expression relative to PKCδ of four independent experiments.

FIG. 4 shows Peroxynitrite decreases ERK phosphorylation. A. CD4+ T cells from normal donors were treated or not for 15 min with peroxynitrite at the indicated concentrations then stimulated with PMA. Cells left untreated were used as control (cont.). Whole cell extracts were fractionated by SDS-PAGE and followed by immunoblot using anti p-T505 PKC δ. After stripping the blot was re-probed with anti p-T²⁰²/Y²⁰⁴-ERK. No variation in total PKCδ or ERK protein expression is observed. This blot is representative of 4 independent experiments. B. Quantitative immunoblot analysis of 4 different experiments. PMA-stimulated non-peroxynitrite treated cells were considered as 1. Values are the mean±SEM.

FIG. 5 shows PKCδ nitration in lupus T cells. CD4+ T cells from three healthy donors (control) were untreated or treated with peroxynitrite followed by PMA stimulation. In parallel CD4+ T cells from six active and four inactive lupus patients were PMA-stimulated. Total lysates were immunoprecipitated then the supernatants and precipitates were immunoblotted with anti-p-T⁵⁰⁵ PKC δ and membranes reprobed with anti-total PKCδ. (A) The bar graph shows the quantitative densitometric analysis of total PKC δ and p-T⁵⁰⁵ PKCδ in the supernatant (spnt) and the precipitate (pp) using CD4+ T cells from healthy donors and patients with active disease. (B) The table shows nitrated PKCδ (total PKCδ content in pp) and p-T⁵⁰⁵PKC δ (p-PKCδ in spnt+pp) as percent of total PKCδ (expressed in arbitrary units) in spnt and pp in each experimental condition. Values are the mean±SEM of six experiments. (C) The graph shows the correlation between the nitrated PKCδ levels in lupus patients with the SLEDAI scores.

FIG. 6 shows a schematic demonstrating the generation of a T cell specific, tet-on dnPKCδ transgenic mouse strain. Mice transgenic for a reverse tetracycline transactivator (rtTA) under the control of a CD2 promoter were crossed with mice transgenic for a dnPKCδ transgene under the control of a tetracycline response element.

FIG. 7 shows a graph demonstrating that doxycycline induces dnPKCδ expression selectively in lymphoid tissue. Mice receiving doxycycline plus sucrose (dark bars) or sucrose alone (light bars) in their drinking water were sacrificed and dnPKCδ mRNA was quantitated relative to GAPDH in the indicated tissues. Significant expression is seen only in the lymph nodes (LN), spleen and thymus.

FIG. 8 shows doxycycline decreases PMA stimulated ERK phosphorylation in CD3+ T cells. Double transgenic tet-on dnPKCδ mice were given dox. FIG. 8A shows a representative immunoblot comparing unstimulated and PMA stimulated ERK phosphorylation in T cells from 3 mice receiving dox to a control receiving just sucrose in the drinking water. FIG. 8B demonstrates a decrease in PMA-stimulated ERK signaling was observed in CD3+ splenic T cells from 4 dnPKCδ/CD2-rtTA mice receiving dox to 4 receiving just sucrose in vivo, and in T cells from 5 mice cultured in vitro with or without dox for 24 h. FIG. 8C shows in vivo and in vitro levels of p-ERK/t-ERK.

FIG. 9 shows a graph depicting DNMT1 expression. FIG. 9 A shows Dnmt1 mRNA levels as measured by real time RT-PCR. FIG. 9B shows decreased Dnmtl levels in double transgenic mice. FIG. 9C shows significantly increased CD70 expression in T cells from dox-treated mice relative to controls.

FIG. 10 shows graphs depicting DNMT1 expression. FIG. 10A shows graphs of relative expression of DNMT. FIG. 10B shows a chart of DNMT1 and CD70 expression in the presence and absence of Doxy treatment.

FIG. 11 shows images depicting IgG deposition on glomeruli of dnPKCδ/CD2rtTA transgenic mice.

FIG. 12 shows images depicting cell infiltration in glomeruli of dnPKC δ/CD2rtTA transgenic mice.

FIG. 13 shows images depicting perivascular infiltration in lung of dnPKC δ/CD2rtTA transgenic mice.

FIG. 14 shows graphs depicting OGT and CXCR levels in hypomethylated CD4+ cells in women/men with lupus as measured by RT-qPCR and normalized to ACTB+18sRNA levels. (A) OGT and (B) CXCR3, isolated CD4+ cells 72 hours following 5AzaC treatment then restimulated with PMA and ionomycin for 6 hours. Data are representative of five independent experiments (n=9 men and 9 women), mean±SEM. (C) and (D), CD4+ cells from women and men with lupus were isolated from lupus patient PBMCs and mRNA levels estimated from RT-qPCR for OGT (C) and CXCR3 (D).

FIG. 15 shows 5-azaC increases OGT protein in CD4 [symbol] T cells from women but not men. PBMC from healthy men and healthy women were stimulated with PHA and cultured for 72 h with or without 5-azaC, restimulated or not with PMA [symbol] ionomycin, then 6 h later CD4 [symbol] T cells were isolated and OGT protein levels measured by immunoblotting. The blots were then stripped and reprobed with anti-actin abs. A. Representative immunoblots of OGT and b-actin in female and male CD4 [symbol] T cells treated with 5-azaC and/or PMA [symbol] ionomycin as indicated. B. Densitometric quantitation of similar OGT and b-actin immunoblots.

FIG. 16 shows 5-azaC demethylates OGT and CXCR3 regulatory elements in CD4 [symbol] T cells from women. PBMC from healthy men and healthy women were stimulated with PHA and treated or not with 5-azaC. DNA was then isolated from CD4 [symbol] Tcells, sonicated into ˜500 bp fragments, methylated fragments affinity purified, and the indicated regions, numbered 50 to the transcription start sites of OGT and CXCR3, amplified by PCR.

FIG. 17 shows OGT and CXCR3 mRNA is overexpressed in CD4 [symbol] T cells from women relative to men with active lupus. CD4 [symbol] T cells were isolated from women (circles) and men (triangles) with inactive and active lupus then (A) OGT or (B) CXCR3 mRNA was measured by RT-qPCR relative to b-actin and 18s-RNA for each subject, and plotted against disease activity as measured by the SLEDAI.

FIG. 18 shows OGT protein levels are increased in CD4 [symbol] T cells from women with active lupus. PBMC were isolated from women with inactive lupus (mean SLEDAI 1.7) and women with active lupus (mean SLEDAI 6.5), stimulated with PMA b ionomycin for 6 h, then CD4 [symbol] T cells were isolated, lysed, and OGT protein measured relative to b-actin by immunoblotting.

FIG. 19 shows OGT and CXCR3 promoters are demethylated in CD4 [symbol] T cells from women with active lupus. DNA was isolated from CD4 [symbol] T cells of 13 women with active lupus, women with inactive lupus and healthy women, and then sonicated into ˜500 bp fragments. Methylated fragments were affinity purified and quantitated by PCR as described in Materials and Methods. Differentially methylated regions of OGT (A) and CXCR3 (B) are numbered relative to the transcription start site.

FIG. 20 shows CBL mRNA levels are increased in CD4 [symbol] T cells from women with lupus. CBL mRNA levels were compared in CD4 [symbol] T cells from healthy women and women with active and inactive lupus.

FIG. 21 shows miR-98 suppresses CBL mRNA. (A) Sequence alignment of human miR-98 and the 30 untranslated region (UTR) of human CBL mRNA, depicting two predicted target sites; site 1 (above, position 6788e6794) and site 2 (below, position 3440e3446). (B) CD4 [symbol] T cells were transfected with a miR-98 mimic or control provided by the manufacturer, then CBL mRNA levels measured relative to b-actin by RT-qPCR. (C) Cytoplasmic proteins were isolated from CD4 [symbol] T cells transfected with the miR-98 mimic or control, and then fractionated by SDS-PAGE and CBL proteins detected by immunoblotting. Controls included probing the filters with anti-actin. (D) CD4 [symbol] T cells from healthy individuals were transfected with the miR-98 mimic or control then CBL protein measured relative to actin by immunoblotting as in panel C, and quantitated by densitometry.

FIG. 22 shows miR-188-3p suppresses CBL mRNA. (A). Sequence alignment of human miR-188-3p and the 30 untranslated region (UTR) of human CBL mRNA, depicting three predicted target sites; site 1 (above, position 117e123), site 2 (middle, position 3175e3181), and site 3 (bottom, position 6279e6285). (B) CD4 [symbol] T cells were transfected with a miR-188-3p mimic or control provided by the manufacturer, and then CBL mRNA levels were measured relative to b-actin by RT-qPCR. (C) Cytoplasmic proteins were isolated from CD4 [symbol] T cells transfected with the miR-188-3p mimic or control, and then fractionated by SDS-PAGE and CBL proteins detected by immunoblotting. Controls included probing the filters with anti-actin. (D) CD4 [symbol] T cells from healthy individuals were transfected with the miR-188-3p mimic or control then CBL protein measured relative to actin by immunoblotting as in panel C, and quantitated by densitometry.

FIG. 23 shows miR-98 and miR-188-3p levels in T cells from women with inactive and active lupus. (A). miR-188-3p levels were measured by RT-qPCR in CD4 [symbol] T cells from women with inactive and active lupus, and results plotted against their SLEDAI scores. (B). miR-98 levels were similarly measured in CD4 [symbol] T cells from the same women.

### DEFINITIONS

As used herein, the term “autoimmune disease” refers generally to diseases which are characterized as having a component of self-recognition. Examples of autoimmune diseases include, but are not limited to, Autoimmune hepatitis, Multiple Sclerosis, Systemic Lupus Erythematosus, Myasthenia Gravis, Type I diabetes, Rheumatoid Arthritis, Psoriasis, Hashimoto's Thyroiditis, Grave's disease, Ankylosing Spondylitis Sjogrens Disease, CREST syndrome, Scleroderma and many more. Most autoimmune diseases are also chronic inflammatory diseases. This is defined as a disease process associated with long-term (>6 months) activation of inflammatory cells (leukocytes). The chronic inflammation leads to damage of patient organs or tissues. Many diseases are chronic inflammatory disorders, but are not know to have an autoimmune basis. For example, Atherosclerosis, Congestive Heart Failure, Crohn's disease, Ulcerative Colitis, Polyarteritis nodosa, Whipple's Disease, Primary Sclerosing Cholangitis and many more.

The clinical manifestations of these diseases range from mild to severe. Mild disease encompasses symptoms that may be function-altering and/or comfort-altering, but are neither immediately organ-threatening nor life-threatening. Severe disease entails organ-threatening and/or life-threatening symptoms. For example, severe autoimmune disease is often associated with clinical manifestations such as nephritis, vasculitis, central nervous system disease, premature atherosclerosis or lung disease, or combinations thereof, which require aggressive treatment and may be associated with premature death. Anti-phospholipid antibody syndrome is often associated with arterial or venous thrombosis.

Diseases can exhibit ranges of activities. As used herein, disease activity (e.g., “active lupus”) refers to whether the pathological manifestations of the disease are fulminant, quiescent, or in a state between these two extremes.

As used here, the term “antibody” includes monoclonal antibodies (including full length monoclonal antibodies), polyclonal antibodies, multispecific antibodies (e.g., bispecific antibodies), and antibody fragments so long as they exhibit the desired biological activity. Antibodies can be conjugated to other molecules.

As used herein, the term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference).

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

The term “biological sample”, as used herein, includes, but is not limited to, samples, such as tissues, cells, whole blood, sera, plasma, saliva, sputa, cerebrospinal fluid, urine, or the like.

As used herein, the term “subject” refers to any animal (e.g., a mammal), including, but not limited to, humans, non-human primates, rodents, and the like, which is to be the recipient of a particular treatment. Typically, the terms “patient” is used interchangeably with reference to a human subject.

As used herein, the term “subject suspected of having autoimmune or chronic inflammatory disease” (e.g., “subject suspected of having lupus”) refers to a subject that presents one or more symptoms indicative of an autoimmune or chronic inflammatory disease (e.g., hives or joint pain) or is being screened for an autoimmune or chronic inflammatory disease (e.g., during a routine physical). A subject suspected of having an autoimmune or chronic inflammatory disease may also have one or more risk factors. A subject suspected of having an autoimmune or chronic inflammatory disease has generally not been tested for autoimmune or chronic inflammatory disease. However, a “subject suspected of having autoimmune or chronic inflammatory disease” encompasses an individual who has received an initial diagnosis but for whom the severity of the autoimmune or chronic inflammatory disease is not known. The term further includes people who once had autoimmune or chronic inflammatory disease but whose symptoms have ameliorated.

As used herein, the term “subject at risk for autoimmune or chronic inflammatory disease” (e.g., “subject at risk for lupus”) refers to a subject with one or more risk factors for developing an autoimmune or chronic inflammatory disease. Risk factors include, but are not limited to, gender, age, genetic predisposition, environmental expose, previous incidents of autoimmune or chronic inflammatory disease, preexisting non-autoimmune or chronic inflammatory diseases, and lifestyle.

As used herein, the term “characterizing autoimmune or chronic inflammatory disease in subject” (e.g., term “characterizing lupus in a subject”) refers to the identification of one or more properties of a sample in a subject, including but not limited to, the presence of calcified tissue and the subject's prognosis. Autoimmune or chronic inflammatory disease (e.g., lupus) may be characterized by the identification of the expression of one or more autoimmune or chronic inflammatory disease marker genes, including but not limited to, markers disclosed herein.

As used herein, the term “autoimmune or chronic inflammatory disease marker genes” (e.g., “lupus marker genes”) refers to a gene whose expression level, oxidation state, methylation status, and/or other characteristic, alone or in combination with other genes/markers, is correlated with autoimmune or chronic inflammatory disease (e.g., lupus) or prognosis of autoimmune or chronic inflammatory disease. The correlation may relate to either increased or decreased expression, an increased or decreased methylation, and/or increased or decreased oxidative state of the gene. Marker expression, methylation, oxidation, and/or other status may be characterized using any suitable method, including but not limited to, those described herein.

As used herein, the term “a reagent that specifically detects expression levels” refers to reagents used to detect the expression of one or more genes (e.g., including but not limited to, the overexpressed lupus markers herein). Examples of suitable reagents include but are not limited to, nucleic acid probes capable of specifically hybridizing to the gene of interest, aptamers, PCR primers capable of specifically amplifying the gene of interest, and antibodies capable of specifically binding to proteins expressed by the gene of interest.

As used herein, the term “initial diagnosis” refers to results of initial autoimmune or chronic inflammatory disease (e.g., lupus) diagnosis. An initial diagnosis does not include information about the severity of the autoimmune or chronic inflammatory disease.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (e.g., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “epigenetic” refers to the chemical marking of the genome and/or proteome as the result of environmental factors, often in combination with genetic facts and/or predispositions. Epigenetic markers include covalent modifications of DNA (e.g. methylation, demethylation), proteins (e.g. nitration, methylation, acetylation, as well as the downstream effects thereof (e.g., changes in protein expression (e.g., overexpression) as the result of alterations in chromosome methylation status).

The terms “overexpression” and “overexpressing” and grammatical equivalents, are used in reference to levels of mRNA to indicate a level of expression approximately 1.5-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

### DETAILED DESCRIPTION

Provided herein are compositions and methods for the diagnosis, monitoring, treatment, and/or prevention of autoimmune disease (e.g., lupus) based on a causal connection with various epigenetic markers (e.g., chromosome demethylation, overexpression of lupus markers, nitration of PKCδ in response to oxidative stress, etc.). In some embodiments, compositions and methods are provided for the diagnosis, monitoring, treatment, and/or prevention of autoimmune disease (e.g., lupus). In particular, a causal connection between epigenetic modifications and lupus is described, and compositions and methods for the diagnosis, treatment, and/or prevention of lupus based thereon are provided. In certain embodiments, the present invention provides epigenetic markers of lupus (e.g., PKCδ nitration (e.g., as the result of oxidative stress), inactive X-chromosome demethylation, overexpression of demethylated genes residing on and related to the X-chromosome (e.g., CD40LG, CXCR3, OGT, miR-98, let-7f-2, miR 188 3p, miR-421, miR-503)) for the treatment, prevention, monitoring, characterization, etc. of lupus, risk of lupus, lupus activity, treatment of lupus, etc.

Experiments were conducted during development of embodiments of the present invention to investigate mechanisms causing the PKCδ phosphorylation defect responsible for decreased ERK pathway signaling in lupus T cells. PKCδ belongs to the PKC family of related serine/threonine kinases with active roles in growth regulation and apoptosis. PKC isoforms contain a highly conserved C-terminal catalytic domain. However, they are subdivided into three subfamilies according to their N-terminal regulatory domains. Conventional isoforms comprise PKC α, β and γ, bind diacylglycerol (DAG)/PMA in their C1 domain, and bind anionic phospholipids in a calcium-dependent manner in their C2 domain. Novel isoforms include PKC δ, ε, η, μ and θ and are activated by DAG/PMA without a calcium requirement. Atypical isoforms, ζ and λ/ι, are DAG/PMA and calcium independent (Nishizuka, 1995; herein incorporated by reference in its entirety).

PKCδ is ubiquitously expressed among cells and tissues, and is the only isoform that can be activated by three different mechanisms: (A) through Ser/Thr phosphorylation, (B) through tyrosine phosphorylation and, (C) by caspase 3-dependent proteolytic cleavage. These are independent mechanisms that regulate PKCδ activity, substrates and cellular localization and play critical roles during cell growth, differentiation, programmed cell death as well as the cellular response to oxidative stress (Steinberg, 2008; herein incorporated by reference in its entirety).

PKCδ p-T⁵⁰⁵ levels are decreased in stimulated lupus T cells, which correlates with decreased ERK pathway signaling in lupus T cells. PKCδ is upstream of ERK, and impaired PKCδ-ERK pathway signaling in T cells causes demethylation and overexpression of methylation sensitive genes (Gorelik, 2007). Transgenic mice lacking T cell PKCδ activity develop a lupus-like disease with decreased ERK signaling, overexpression of methylation sensitive genes and production of anti dsDNA antibodies similar to those observed in lupus patients (Gorelik et al., Lupus 19:7, 2010; herein incorporated by reference in its entirety), strongly indicating that defective PKCδ signaling is sufficient to cause lupus.

PDK1 phosphorylates PKCδ on T⁵⁰⁵ in the activation loop, promoting alignment of these residues with the catalytic pocket and controlling catalytic activity of the enzyme (Le Good, 1998; herein incorporated by reference in its entirety). Phosphorylation of Ser²⁴¹, required for PDK-1 kinase activity, is not appreciably affected in lupus T cells relative to T cells from healthy donors and under the same assay conditions in which PKC δ p-T⁵⁰⁵ was decreased. This implies that another mechanism inhibits PKC δ T⁵⁰⁵ phosphorylation in lupus T cells.

Experiments were conducted during development of embodiments of the present invention to determine whether defective PKCδ activation in lupus T cells was caused by oxidative damage. ONOO was used as the oxidizing agent, and caused PKCδ nitration that resulted in decreased phosphorylation of T⁵⁰⁵ in. The fact that PKCδ was the only PKC isoform catalytically affected indicates that the ONOO⁻ inhibitory effect is selective and specific to PMA-stimulated PKCδ p-T⁵⁰⁵. PKCδ is not phosphorylated at the activation loop (T⁵⁰⁵) in resting T cells, but phosphorylation increases following PMA stimulation and translocation to the cytoplasmic membrane. Oxidation modified the phosphorylation pattern, increasing tyrosine phosphorylation while decreasing threonine phosphorylation, indicating specific effects on PKCδ phosphorylation regulation. This differential PKCδ phosphorylation pattern also modifies its intracellular translocation, resulting in changes to the interaction of PKCδ with downstream targets (Rybin, 2004; herein incorporated by reference in its entirety). The present work shows that oxidation of PKCδ results in a selective decrease in PKCδ p-T⁵⁰⁵ and directly correlates with decreased p-ERK in T cells.

Experiments conducted during development of embodiments described herein demonstrate higher level of nitrotyrosine-containing proteins in T cells from patients with active lupus relative to patients with inactive lupus and to T cells from healthy controls. The levels of nitrated PKCδ were also greater in T cells from patients with active lupus, and PMA stimulated T⁵⁰⁵ phosphorylation of the nitrated proteins was impaired. Similar results were observed in control T cells pretreated with ONOO—.

Experiments conducted during development of embodiments of the present invention reveal that the impaired T cell PKCδ kinase activation observed in patients with active lupus is due to the oxidative damage, and causes the impaired ERK pathway signaling in T cells from lupus patients. These studies point to PKCδ as a link between oxidative stress, caused by environmental agents, and the epigenetic changes observed in lupus T cells. Other poorly understood autoimmune diseases also result from gene-environment interactions, and involve epigenetic mechanisms that include not only DNA methylation but histone modifications and microRNA's as well (Hedrich, 2011; herein incorporated by reference in its entirety). Oxidative stress may also contribute to these disorders through mechanisms such as altered signaling.

OGT catalyzes the transfer of N-acetylglucosamine (GlcNAc) from UDP-N-acetylglucosamine to serines and threonines in cytoplasmic and nuclear proteins to form O-linked β-N-acetylglucosamine (O-GlcNAc). In contrast to cell surface carbohydrates, O-GlcNAc is not elongated into more complex structures. O-GlcNAc serves as a signaling molecule, and OGT effects are opposed by β-N-acetylglucosaminidase (NAGA), which removes GlcNAc in a cyclic manner analogous to protein phosphorylation and dephosphorylation. Also analogous to phosphorylation, O-GlcNAcylation alters the posttranslational fate and function of proteins. Further, there are complex interactions between GlcNAc and phosphate that include competition for the same sites in some proteins such as c-Myc and eNOS. This cycling of GlcNAc residues provides a signaling mechanism referred to as the “hexosamine signaling pathway” (HSP), and is found in all metazoans but not yeast. Since GlcNAc derives from glucose, the serves in part as a nutrient sensor, indicating an environmental linkage to this signaling pathway. OGT levels also increase in response to multiple forms of cellular stress, including UV light, heat and ethanol, rendering cells more thermotolerant, providing another environmental link.

The presence of a second X chromosome in women (XX) has been implicated in female predisposition to lupus. Men with Klinefelter's syndrome with an additional X chromosome (XXY), have a 14 fold increase over general population to develop lupus (Scofield, 2008; herein incorporated by reference in its entirety). Almost 90% second copy of the X chromosome encoded genes in inactive X chromosome are silenced in general female population. DNA hypermethylation, and histone acetylation/methylation status and the coating of X-inactive specific transcript (Xist) mediate the silencing of genes on inactive X (Xi). Experiment conducted during development of embodiments of the present invention indicate that escape of inactivation due to DNA demethylation results in overexpression of immune activating genes linked to human lupus. (Strickland, 2012; Pisitkun, 2006; herein incorporated by reference in their entireties).

Experiments conducted during development of embodiments of the present invention demonstrate that DNA hypomethylation causes re-activation and subsequent over-expression of genes involved in lupus pathogenesis. CD40LG CD4+ T cells from patients with SLE have hypomethylated DNA, and the degree of DNA demethylation correlates inversely with disease activity. Experiments conducted during development of embodiments of the present invention demonstrated that OGT, CXCR3 mRNAs and 5 miRNAs overexpressed in female lupus patients compared with healthy control as well as male lupus patients. Evidence demonstrates that female biased overexpression is a result of demethylation mediated reactivation from inactive X. 5AzaC treatment is sufficient to reactivate the transcription in female CD4+ cells, which indicates that DNA methylation is a primary mechanism in silencing these genes in inactive X. Experiments conducted during development of embodiments of the present invention identified several hypomethylated sites linked to the overexpressed genes in women with lupus compared with healthy women. These methylation sensitive sites comprise of multiple regulatory element binding matrices of promoter modules. These promoter modules may exhibit synergistic, antagonistic or additive functions in steering the actual transcription process. Hypomethylation of these sites can enhance the binding of some of these TFs to reactivate the expression from inactive X.

The OGT gene encodes a glycosyltransferase that catalyzes the O-linked 13-N-acetylglucosamine (O-GlcNAc) posttranslational modification to serine or threonine residues in a variety of proteins including signaling molecules and TFs. OGT is overexpressed in CD4+ cells from 5AzaC induced demethylation and lupus patients. In lupus patients, the overexpression is correlated with the disease activity. Furthermore, OGT mRNA levels are significantly higher in women with lupus compared with men with lupus, suggesting a contribution from Xi. Experiments conducted during development of embodiments of the present invention identified DNA hypomethylation sites on the OGT promoter region where, the in silico analysis demonstrated the existence of TF binding sites. Of these, existence of STAT binding sties is of particular interest as STAT play an essential role in transmitting cytokine mediated signaling and Th cell differentiation. The hypomethylation of the X, permits these regions to be accessible to STAT binding and to upregulate OGT transcription in T cell activation process. Many intracellular signaling pathways are influenced from protein O-glycosylation by OGT. Alterations in these modifying signaling due to OGT signaling contributes to pathogenic status of the hyperactive T cells in lupus.

The CXCR3 gene encodes a G protein-coupled (C-X-C motif) receptor 3. CXCR3 can bind interferon inducible chemokines; CXCL9, CXCL10 and CXCL11. Chemokine binding to CXCR3 induces leukocyte trafficking to inflamed tissues. CXCR3 has been implicated in mediating Th1 and Th17 immune response. Similar to OGT, CXCR3 is mRNA is elevated in 5AzaC treated and lupus CD4+ cells. CXCR3 expression in women with lupus is significantly higher than men with lupus. As in OGT, DNA hypomethylation site of CXCR3 consists of STAT binding sites. Furthermore, CXCR3 methylation sensitive site can bind EREF TFs too. EREF binding to hypomethylated X, can influence the transcription. In addition to demethylation from Xi, EREF binding provides an additive effect to female predisposition to lupus.

Experiments were conducted during development of embodiments of the present invention to characterize the relationships between miRNAs, DNA methylation, and CD4+ T cell activity in humans with lupus. LET&7F-2*, MIR98, MIR188-3p, MIR421 and MIR503 were overexpressed in demethylated and lupus T cells. Single miRNA can target multiple mRNAs and multiple miRNAs can target one mRNA. The functional consequences of the overexpression miRNAs was assessed using bioinformatics tools, and then the molecular pathways affected by overexpressed miRNAs. The biological target genes which steer immune regulatory mechanisms in T cells were identified.

Experiments were conducted during development of embodiments of the present invention demonstrated that MIR98 is over-expressed in both hypomethylated CD4+ T cells and in CD4+ T cells from lupus patients. MIR98/LET7F-2* cluster is encoded on an intron of protein coding gene HUWE1. A region upstream of MIR98 was identified which contains STAT and ETSF binding matrices as common to OGT and CXCR3 methylation sensitive sites, and may regulate the expression of MIR98 or the host genes HUWE1. MIR98 has been shown to target FAS and regulate activation induced cell death process. E3 ubiquitin ligase CBLC is a protein that negatively regulates pro-inflammatory pathways by modulating TCR signaling. CBLC is decreased in CD4+ T cells from lupus patients and Cbl-c deficient mouse develop a lupus-like disease. Pursuing a bioinformatics-based prediction that MIR98 could also block CBLC, we first showed that CBLC levels are decreased in hypomethylated CD4+ T cells. Experiments were conducted during development of embodiments of the present invention demonstrated that MIR98 over-expression blocks expression of CBLC in CD4+ T cells from healthy donors.

In addition to CBLC and FAS, TragetScan analysis revealed that MIR98 is predicted to target CASP3. CASP3 is involved in maintaining T cell unresponsiveness by cleaving T cell activating molecules VAV1 and GRB2. Similarly, other methylation sensitive miRNAs too, can play a role in suppressing immune modulatory molecules. MIR421 was predicted to target CASP3 and GRAIL. Furthermore, tumor suppressor protein tyrosine phosphatase PTEN, which negatively regulate PI3K pathway, is predicted to be a target of MIR188.

Experiments conducted during development of embodiments of the present invention demonstrate that DNA demethylation of the X, contributes to overexpression of OGT, CXCR3, and miRNAs encoded on X chromosome. OGT through protein O-glycosylation, and CXCR3 through leukocyte trafficking contributes to autoimmune response. MIR98 overexpression can decrease the level of immune modulators such as CBLC, CASP3, FAS etc., which result in T cell hyperactivity and autoimmune response. Similarly other X chromosome encoded methylation sensitive miRNA overexpression downregulate multiple immunomodulatory molecules responsible for controlling TCR signaling cascade.

The present invention provides methods of determining the presence and/or levels of various epigenetic markers. In certain embodiments, method for determining the presence and/or levels of general or select oxidation products (e.g., oxidized PKCδ, PKCδ oxidized at specific amino acids) are provided. In other embodiments, methods of detecting and/or quantitating chromosome (e.g., X-chromosome (e.g., inactive X-chromosome)) demethylation are provided. In some embodiments, methods are provided for determining the methylation status of chromosomes (e.g., X-chromosome (e.g., inactive X-chromosome)). Any suitable methods for determining the presence and/or levels of various epigenetic markers are within the scope of the present invention. For example, in some embodiments, levels of markers (e.g., gene expression) are determined by, ELISA, Western blot, quantitative immunfluorescence, mass spectrometry, etc.

Dityrosine and nitrotyrosine levels in the biological sample can be determined using monoclonal antibodies that are reactive with such tyrosine species. For example, anti-nitrotyrosine antibodies may be made and labeled using standard procedures and then employed in immunoassays to detect the presence of free or peptide-bound nitrotyrosine in the sample. Suitable immunoassays include, by way of example, radioimmunoassays, both solid and liquid phase, fluorescence-linked assays or enzyme-linked immunosorbent assays. Preferably, the immunoassays are also used to quantify the amount of the tyrosine species that is present in the sample.

Monoclonal antibodies raised against the dityrosine and nitrotyrosine species are produced according to established procedures. Generally, the dityrosine or nitrotyrosine residue, which is known as a hapten, is first conjugated to a carrier protein and used to immunize a host animal. Preferably, the dityrosine and nitrotyrosine residue is inserted into synthetic peptides with different surrounding sequence and then coupled to carrier proteins. By rotating the sequence surrounding the dityrosine and nitrotyrosine species within the peptide coupled to the carrier, antibodies to only the dityrosine and nitrotyrosine species, regardless of the surrounding sequence context, are generated. Similar strategies have been successfully employed with a variety of other low molecular weight amino acid analogues.

Suitable host animals, include, but are not limited to, rabbits, mice, rats, goats, and guinea pigs. Various adjuvants may be used to increase the immunological response in the host animal. The adjuvant used depends, at least in part, on the host species. To increase the likelihood that monoclonal antibodies specific to the dityrosine and nitrotyrosine are produced, the peptide containing the respective dityrosine and nitrotyrosine species may be conjugated to a carrier protein which is present in the animal immunized. For example, guinea pig albumin is commonly used as a carrier for immunizations in guinea pigs. Such animals produce heterogenous populations of antibody molecules, which are referred to as polyclonal antibodies and which may be derived from the sera of the immunized animals.

Monoclonal antibodies, which are homogenous populations of an antibody that binds to a particular antigen, are obtained from continuous cells lines. Conventional techniques for producing monoclonal antibodies are the hybridoma technique of Kohler and Millstein (Nature 356:495-497 (1975)) and the human B-cell hybridoma technique of Kosbor et al. (Immunology Today 4:72 (1983)). Such antibodies may be of any immunoglobulin class including IgG, IgM, IgE, Iga, IgD and any class thereof. Procedures for preparing antibodies against modified amino acids, such as for example, 3-nitrotyrosine are described in Ye, Y. Z., M. Strong, Z. Q. Huang, and J. S. Beckman. 1996. Antibodies that recognize nitrotyrosine. Methods Enzymol. 269:201-209.

In general, techniques for direct measurement of protein bound dityrosine and nitrotyrosine species from biological fluids involves removal of protein and lipids to provide a fluid extract containing free amino acid residues. The tissues and biological fluids are stored, preferably in buffered, chelated and antioxidant-protected solutions. The frozen tissue, and biological fluids are then thawed, homogenized and extracted to remove lipids and salts. Heavy isotope labeled internal standards are added to the pellet. The sample is then derivatized and analyzed by stable isotope dilution gas chromatography-mass spectrometry as above. Values of free dityrosine and nitrotyrosine species in the biological sample can be normalized to protein content, or an amino acid such as tyrosine.

In some embodiments, mass spectrometry methods are used for the measurement of oxidative products (e.g., nitrotyrosine and dityrosine).

In some embodiments, autoimmune risk factors described herein are compared to a control, threshold, or predetermined value. The predetermined value is based upon the levels of risk factor in comparable samples obtained from the general population, from a select population of human subjects (e.g., those without the autoimmune disease), or from the specific individual when not suffering from the disease. The select population may be comprised of apparently healthy subjects. “Apparently healthy”, as used herein, means individuals who have not previously had any signs or symptoms indicating autoimmune disease, and/or has not received a diagnosis of autoimmune disease. Apparently healthy individuals also do not otherwise exhibit symptoms of disease. In other words, such individuals, if examined by a medical professional, would be characterized as healthy and free of symptoms of disease.

The predetermined value is related to the value used to characterize the level of the risk factor (e.g., methylation status of X-chromosome, expression level of marker genes, level of oxidative modification of PKCδ) in the biological sample obtained from the test subject. The predetermined value can take a variety of forms. The predetermined value can be a single cut-off value, such as a median or mean. The predetermined value can be established based upon comparative groups such as where the risk in one defined group is double the risk in another defined group. The predetermined can be a range, for example, where the general population is divided equally (or unequally) into groups, such as a low risk group, a medium risk group and a high-risk group, or into quadrants, the lowest quadrant being individuals with the lowest risk the highest quadrant being individuals with the highest risk.

Experiments were conducted during development of embodiments of the present invention to develop a strain of transgenic mice resulting from breeding mice that express dnPKCδ under the control of a tetracycline-dependent promoter, and mice in which a reverse tetracycline-controlled transactivator is expressed under the control of a CD2 promoter. This generated a double transgenic mouse that responds to Doxycycline with transcription of the dnPKC6 gene specifically in T cells. This mouse model provides an inducible lupus-like state for the study of lupus, screening lupus therapies (e.g., therapeutics) and the identification/validation of lupus biomarkers (e.g., epigenetic markers).

### EXPERIMENTAL

### Example 1

### Materials and Methods

### Examples 2-6

### Reagents.

Hydralazine was purchased from VWR (West Chester, Pa.), and peroxynitrite from Calbiochem (Gibbstown, N.J.). All other chemicals were from Sigma.

### Antibodies.

The following primary antibodies were used: polyclonal rabbit anti-phospho-PKCα (T^{638/641}), anti-phospho-PKCθ (T⁵³⁸), anti-phospho-PKCδ (T⁵⁰⁵), anti-phospho-PKCδ (Y³¹¹) and anti-phospho-PDK1 (Ser²⁴¹), at 1:1000 dilution (Cell Signaling Tech., Beverly, Mass.).

For immunoprecipitation, anti-nitrotyrosine, clone 1A6 agarose conjugate, was used (Upstate-Millipore, Billerica, Mass.). Rabbit polyclonal anti-active MAPK (1:5000) was from Promega (Madison Wis.), and anti-total PKCδ was from Santa Cruz Biotechnology (Santa Cruz, Calif.). Secondary antibodies included: anti-rabbit IgG horseradish peroxidase (1:2000, Cell Signaling Technology, Danvers, Mass.) and anti-mouse IgG horseradish peroxidase (1:4000, Amersham, Piscataway, N.J.).

### Subjects.

Lupus patients (average age 42 range 27-64 years) with active and inactive disease were recruited from the outpatient rheumatology clinics and inpatient services at the University of Michigan, and healthy controls were recruited by advertising. All lupus patients met the revised American College of Rheumatology criteria for SLE (Tan, 1983; herein incorporated by reference in its entirety). Lupus disease activity was quantitated using the systemic lupus erythematosus disease activity index (SLEDAI) (Bombardier, 1992; herein incorporated by reference in its entirety), and the range was 4-10 (mean 6.2) for the patients with active lupus, and 0-2 (mean 0.5) for patients with inactive disease. Controls were matched to the lupus patients for age, race and sex. These protocols were reviewed and approved by the University of Michigan Institutional Review Board for Human Subject Research. The demographics and medications received by the patients are summarized in Table 1.

TABLE 1

| | Patients |
|---|---|
| SLEDAI (mean ± S.D.) | |
| Active | 6.2 ± 2.3 |
| Inactive | 0.5 ± 1.0 |
| Age, years (mean ± S.D.) | 41.8 ± 11.1 |
| Gender, ratio (F:M) | 14:2 |
| Medications: | (%) |
| Prednisone | 62 |
| Antimalarials | 62 |
| Azathioprine | 25 |
| Mycophenolate mofetil | 44 |
| Methotrexate | 0 |

### T Cell Isolation.

Peripheral blood mononuclear cells were isolated from healthy donors or SLE patients by Ficoll-Hypaque density gradient centrifugation. CD4+ T cells were then purified by negative selection using magnetic beads (CD4+ T cell isolation kit; Miltenyi Biotec, Auburn, Calif.) as previously reported (Gorelik, 2007; herein incorporated by reference in its entirety).

### T Cell Stimulation and Protein Isolation.

CD4+ T cells were suspended in RPMI 1640 supplemented with 10% fetal calf serum, 2 mM glutamine and penicillin/streptomycin then left unstimulated or stimulated with 50 ng/ml PMA for 15 min at 37° C. Treatment with peroxynitrite was performed at the concentrations and time specified in each experiment and before PMA stimulation. Following stimulation, whole cell lysates were obtained and protein content quantified using the BCA Protein Assay (Pierce, Rockford, Ill.).

### Transient Transfections.

T cells from normal donors were immediately transfected with siRNA or the indicated mutants using Amaxa nucleofection technology (Gaithersburg, Md.). After 24 h the cells were treated as indicated, harvested and cell lysates obtained as above. siRNA PP2Ac and PP2Ac mutants H118N and L199P were used (Katsiari, 2005; herein incorporated by reference in its entirety). Transfection efficiency was 63±6% of total cell number and verified by fluorescence microscopy of cells transfected with the positive control vector pmaxGFP encoding a green fluorescence protein and provided with the kit.

### Immunoblot Analyses.

Whole cell protein was fractionated by SDS-PAGE, transferred to nitrocellulose membranes (Schleicher and Schuell, Keene, N.H.) then stained with Ponceau S (Sigma) to verify equal protein loading between lanes. After incubation with the kinase specific antibody then the secondary antibody, protein bands were visualized by chemiluminescence (Amersham, Piscataway, N.J.). The bands on X-ray films were scanned and analyzed with ImageQuant 5.2 software (Amersham) for quantification. Where indicated, blots were stripped and re-probed with the corresponding antibody. Values were normalized to β-actin or the corresponding kinase as indicated.

### Immunoprecipitations.

Immunoprecipitations were performed according to the manufacturer's instructions. Lysates from normal or lupus T cells were incubated with the agarose-conjugated anti-nitrotyrosine antibody overnight at 4° C. Following centrifugation an aliquot of the supernatant (containing non-nitrated proteins) and the beads were resuspended in Laemmli sample buffer and boiled for 5 min. Proteins from each fraction were then analyzed in parallel by SDS-PAGE and immunoblot analysis.

### Example 2

### PDK-1 does not Contribute to the PKCδ Signaling Defect in Lupus T Cells

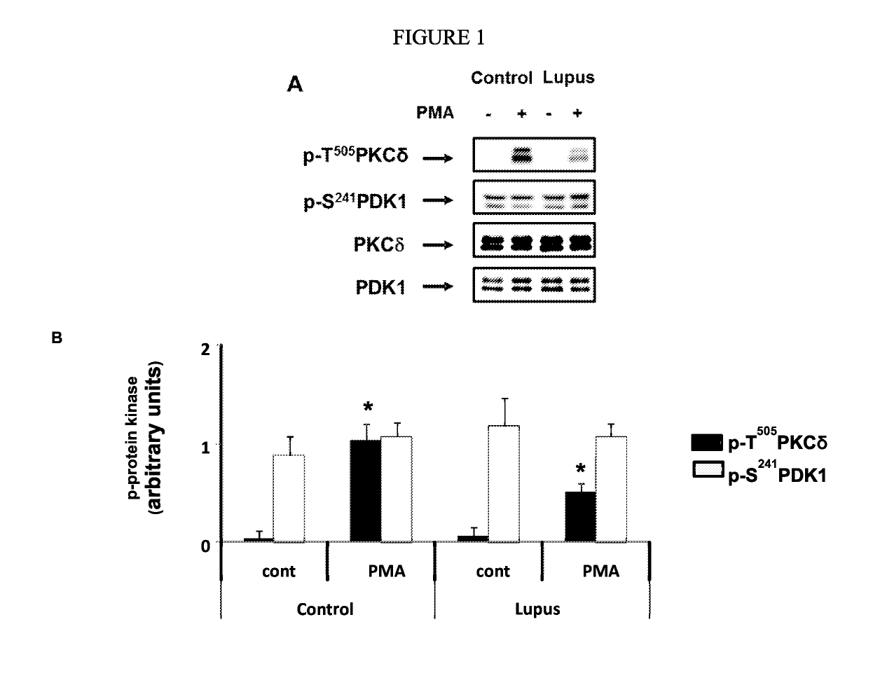

3′-Phosphoinositide-dependent protein kinase-1 (PDK-1) controls phosphorylation of T505 in the PKCδ catalytic loop (Le Good, 1998; herein incorporated by reference in its entirety). PDK-1 has five sites (Ser-25, Ser-393, Ser-396, Ser-410 and Ser-241) that are phosphorylated, but only Ser-241 is located in the activation loop and required for PDK-1 activity (Casamayor, 1999). PDK-1 activation was therefore studied using antibodies to PDK-1 p-Ser²⁴¹ and immunoblotting. There was no significant difference in PDK-1 phosphorylation between control and lupus T cells, although PKCδ p-T⁵⁰⁵ was decreased in PMA-stimulated CD4+ T cells from lupus patients with active disease (FIG. 1A). The densitometric analysis of four similar experiments confirmed no significant difference in PDK-1 activation between lupus patients and controls (FIG. 1B). The observation that p-PDK-1 is expressed at similar levels in resting and PMA-stimulated T cells agrees with the observation that PDK-1 is constitutively active due to autophosphorylation of its activation loop (Knight, 2011; herein incorporated by reference in its entirety). These results indicate that a PDK-1 activity defect is unlikely causing the decreased PKCδ T⁵⁰⁵ phosphorylation in CD4+ lupus T cells.

### Example 3

### ONOO— Inhibits PKCδ T₅₀₅ Phosphorylation

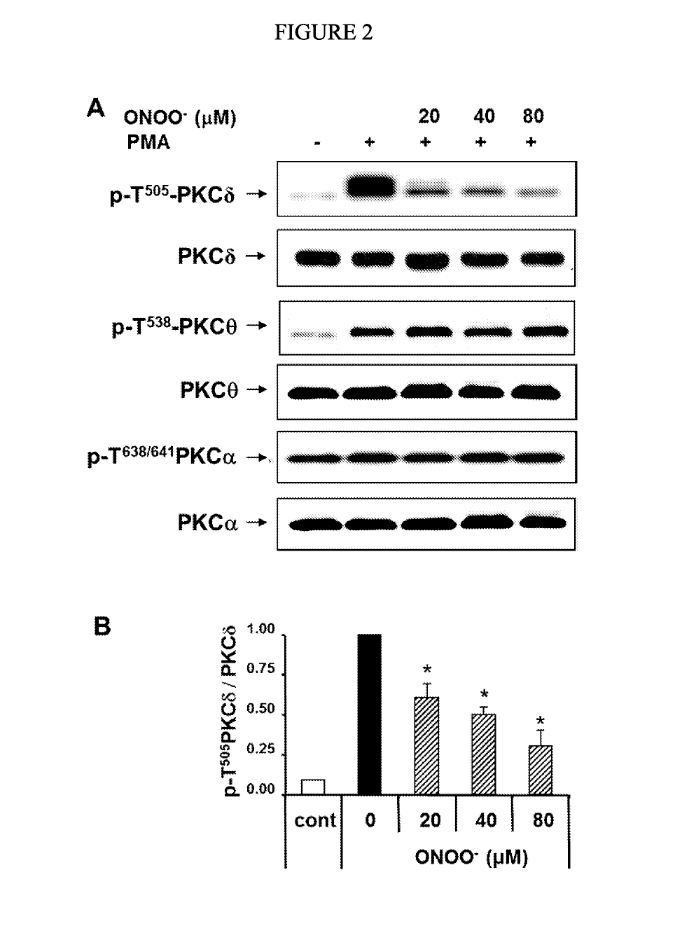

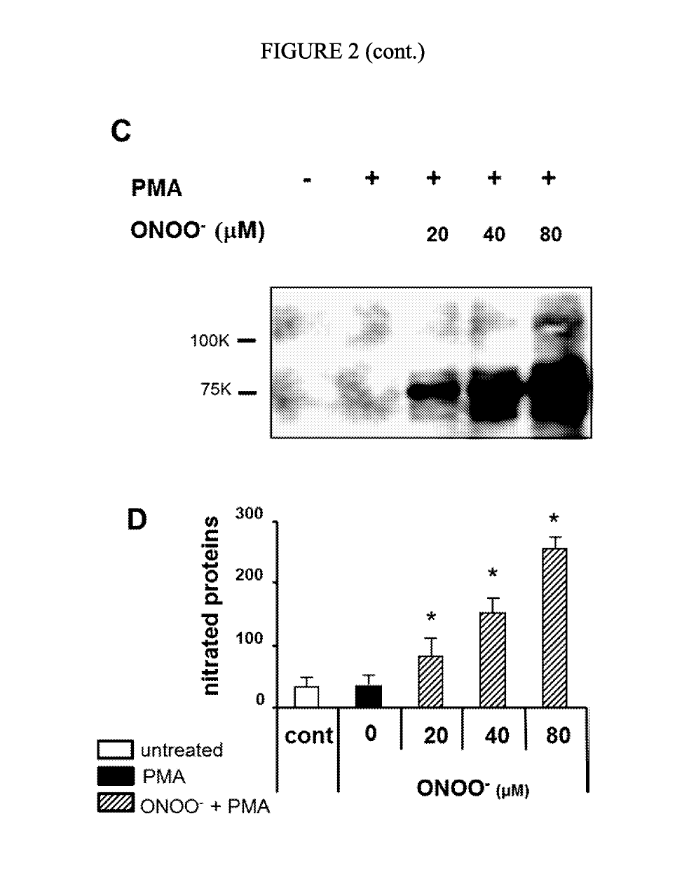

Experiments were performed during development of embodiments of the present invention to determine if ONOO⁻ causes the PKCδ T⁵⁰⁵ phosphorylation defect observed in lupus T cells. CD4+ T cells from healthy subjects were treated with increasing concentrations of ONOO⁻ then stimulated with PMA. Treatment with ONOO⁻ caused a concentration dependent decrease in PKCδ T505 phosphorylation while PKC α and θ activation loop phosphorylation was not appreciably affected (FIG. 2A). FIG. 2B shows the quantitative densitometric analysis of four serial repeat experiments similarly measuring the effects of ONOO⁻ on PKCδ p-T⁵⁰⁵. These results indicate that the inhibitory effects of ONOO⁻ on PKCδ are isoform-specific since phosphorylation of other isoforms was unaffected.

Experiments were conducted during development of embodiments of the present invention to confirm the effect of ONOO⁻ by measuring 3NO₂-tyrosine protein derivative formation. CD4+ T cells from a healthy donor were treated with increasing ONOO— concentrations then stimulated with PMA. 3NO₂-tyrosine modified proteins were studied by immunoblotting. A concentration-dependent increase in 3-nitrotyrosine derivative formation was observed (FIG. 2C). FIG. 2D shows the mean±SD of densitometric analyses from four similar experiments, confirming that T cell proteins are nitrated by ONOO⁻ and their nitration levels correlate with the ONOO⁻ induced inhibition of p-T⁵⁰⁵ PKCδ shown in FIG. 2B.

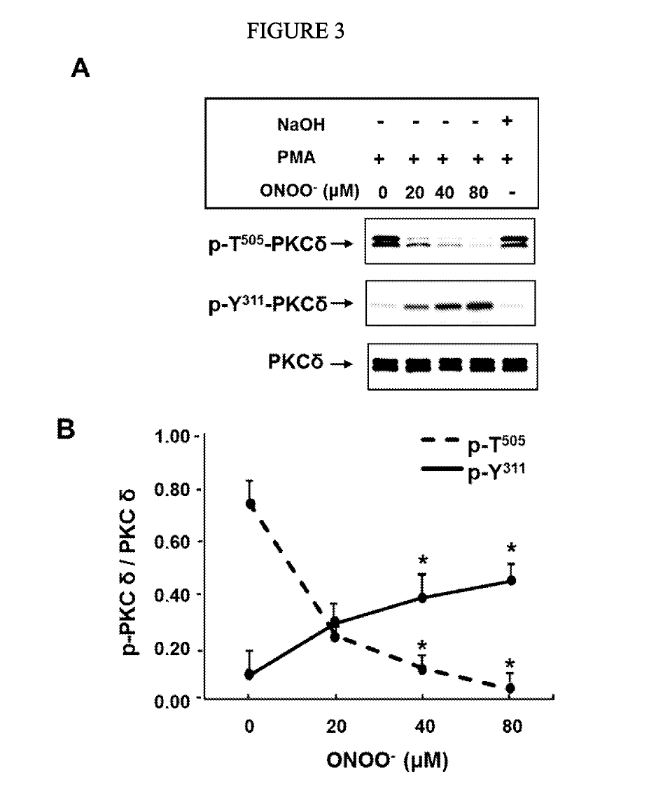

The phosphorylation of Y³¹¹ in the PKCδ molecule was also examined, because this amino acid is phosphorylated in cells undergoing oxidative stress (Steinberg, 2004; Kikkawa, 2002; herein incorporated by reference in their entireties). FIG. 3A shows a representative immunoblot comparing effects of ONOO— on PMA stimulated PKCδ T⁵⁰⁵ and Y³¹¹ phosphorylation. In untreated (0 μM ONOO⁻) CD4+ T cells, PMA causes a substantial increase in T⁵⁰⁵ phosphorylation, but has no appreciable effect on Y³¹¹ phosphorylation. It is important to note that p-Y³¹¹ is almost undetectable in resting CD4+ T cells (not shown) similar to p-T⁵⁰⁵. However, ONOO⁻ induces a concentration-dependent increase in Y³¹¹ phosphorylation in PMA stimulated cells that is inversely related to T505 phosphorylation (ONOO⁻ vs non-ONOO⁻). FIG. 3B clearly demonstrates a different pattern of PKCδ phosphorylation that is stimulus dependent. While PMA promotes phosphorylation on T⁵⁰⁵, Y³¹¹ is almost insensitive. In contrast, ONOO⁻ increases Y311 phosphorylation with a concomitant decrease in p-T⁵⁰⁵.

The lack of additional effects caused by NaOH excludes nonspecific effects due to pH changes caused by the ONOO⁻ solution (FIG. 3A).

### Example 4

### Protein Phosphatase 2Ac (PP2Ac) does not Participate in the ONOO⁻ Induced PKCδ Defect

Experiments were conducted during development of embodiments of the present invention to determine if increased PP2Ac activity could be responsible for the PKCδ dephosphorylation caused by ONOO. T cells from healthy donors were transfected with 5 μg of plasmids encoding the dominant negative PP2Ac mutants H118N or L199P (32), or 100 nM PP2Ac-specific siRNA according to Katsiari et al. Cells transfected with an irrelevant siRNA were used as control and 2 μg of the empty GFP vector were transfected as an internal control for transfection efficiency in each experiment. Twenty four hours later the cells were treated with ONOO for 15 min followed by PMA-stimulation. In three serial repeats, PKCδ T⁵⁰⁵ phosphorylation was not increased in ONOO treated cells overexpressing the catalytically inactive PP2Ac mutants or the siRNA relative to non-transfected cells relative to PMA-stimulated T cells (100%), confirming that PP2Ac is not causing the p-PKCδ T⁵⁰⁵ defect in this system.

### Example 5

### PKCδ Nitration Correlates with Decreased ERK Phosphorylation

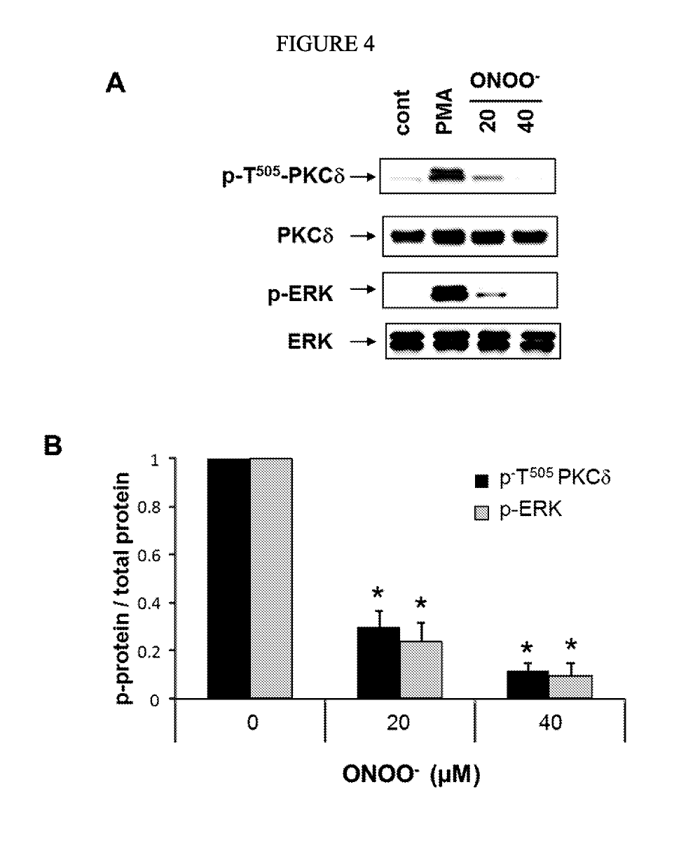

To determine if PKCδ nitration also inhibits ERK phosphorylation, CD4+ T cells from healthy controls were treated with different concentrations of ONOO— for varying times then stimulated with PMA. FIG. 4A shows that as expected, PMA-stimulated ERK phosphorylation declines with increasing ONOO— concentrations, and parallels the decrease in PKCδ T505 phosphorylation. This agrees with our previous results showing that PKCδ T505 phosphorylation is upstream of ERK (Gorelik, 2007; herein incorporated by reference in its entirety) and its impairment decreases ERK pathway signaling. FIG. 4B shows the quantification of 4 serial repeats confirming this observation.

### Example 6

### PKCδ Nitration in Lupus

Experiments were conducted during development of embodiments of the present invention to determine whether PKCδ is inactivated in T cells from patients with active lupus through nitration, similar to ONOO treated T cells. CD4+ T cells from 6 lupus patients with active disease (SLEDAI≧4) were stimulated with PMA and nitrated proteins immunoprecipitated from the lysates with anti-3NO₂-Tyr antibodies. Controls included similarly treated CD4+ T cells from 4 lupus patients with inactive disease, and 3 control subjects. Total and T505 phosphorylated PKCδ content were then compared by immunoblotting.

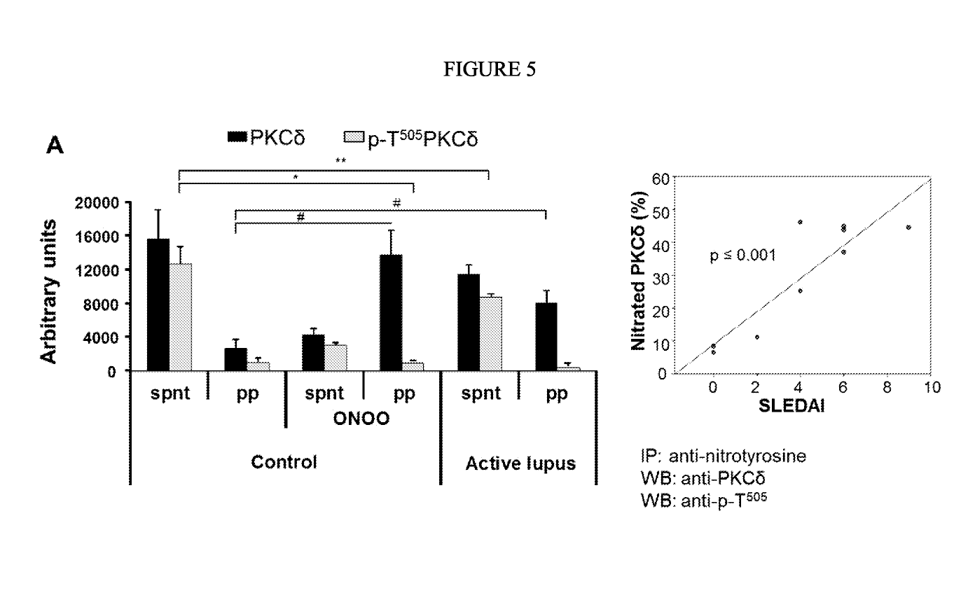

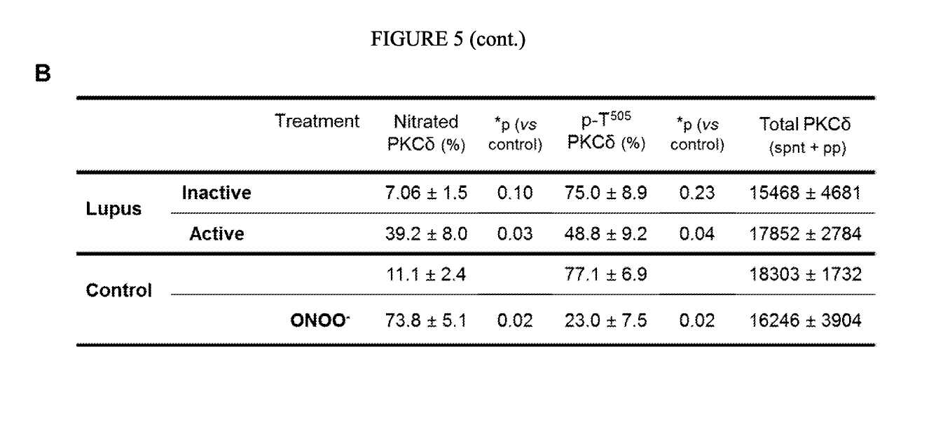

Nitrated and non-nitrated PKCδ were analyzed as the total PKCδ content in the precipitate and supernatant respectively. The levels of total PKCδ (supernatant+immunoprecipitate) were similar in untreated or ONOO⁻ treated T cells from control donors and lupus patients (FIGS. 5 A and B). These results demonstrate no differences in PKCδ protein expression in control and lupus patients (Gorelik, 2007; herein incorporated by reference in its entirety). The amount of nitrated PKCδ (precipitated) was higher in T cells from patients with active lupus relative to controls and with a pattern similar to ONOO⁻ treated control T cells (FIG. 5A). In contrast, FIG. 5B shows that the pattern of nitration and phosphorylation of PKCδ was not significantly different in CD4+ T cells from patients with inactive lupus when compared to healthy controls. Importantly, the level of T cell PKCδ nitration in lupus patients was directly correlated with the SLEDAI scores (FIG. 5C).

However, overall PKCδ T505 phosphorylation was decreased in stimulated T cells from patients with active lupus relative to healthy controls (FIG. 5B), and the nitrated PKCδ fraction from the active lupus patients was almost completely refractory to PMA-stimulated phosphorylation (4±2% of nitrated PKCδ, p<0.04, active lupus vs control, FIG. 5A). As predicted, 74% of the PKCδ in T cells treated with ONOO— was nitrated but only 6% of this fraction was T505 phosphorylated after PMA stimulation (FIG. 5A). In contrast, non-nitrated PKCδ was phosphorylated in both control and active or inactive lupus T cells (supernatant) (FIG. 5A). These experiments thus demonstrate more extensive PKCδ nitration, and less T505 phosphorylation, in T cells from patients with active lupus relative to both inactive lupus and control, suggesting that T505 phosphorylation decreases in proportion to the extent of nitration and that the degree of nitration is directly related to the disease activity.

### Example 7

### Materials and Methods

### Examples 8-13

Generation of a dnPKCδ/PCR2.1 Construct

A dnPKCδ cDNA was PCR amplified from a plasmid encoding a dominant negative PKC-δ^{K3768}-pEGFP-N1fusion protein, using primers with an EcoR1 restriction site at the 5′ end and a BamH1 site at the 3′ end. A stop codon was added to the 3′ end, using High Fidelity Taq polymerase (Roche). “A” overhangs were added using Taq polymerase (Invitrogen) then the construct was subcloned into the PCR 2.1 vector using TA cloning method. The entire sequence was verified, and confirmed the K³⁷⁶R mutation and the absence of any other PCR induced base changes.

Generation of a dnPKC5/pTRE-Tight Construct and Transgene

The dnPKCδ was excised from the dnPKCδ/PCR 2.1 construct using EcoR1 and BamH1 then ligated into pTRE-Tight. Subcloning was confirmed by sequencing. The dnPKCδ/pTRE-Tight construct was then digested with Xho1 to excise the dnPKCδ along with the tet-on promoter and the poly A tail for microinjection.

### Generation of Transgenic Mice

Double transgenic mice were generated and maintained in a specific pathogen-free environment. All protocols were approved by the University of Michigan Committee on the Use and Care of Animals (UCUCA). Pups were weaned at 20 days of age and genotyped for the presence of both genes by PCR. Transgene expression was induced by giving 2 mg/ml of dox in the drinking water and supplemented with 5% of sucrose. Double transgenic control animals were given 5% sucrose alone. All the double transgenic mice were developmentally normal. Protein and blood in mouse urine was measured using Chemstrip 6 dipsticks (Roche, Madison, Wis.).

### Cell Purification and Culture

CD4+ T cells were isolated from the spleens of transgenic mice using magnetic beads (Miltenyi Biotec, Auburn Calif.) and negative selection. Where indicated, the cells were cultured in RPMI 1640 supplemented with 10% fetal calf serum, 2 mM glutamine and penicillin/streptomycin, without or with doxycycline (2 μg/ml) for 24 h. Where indicated, the cells were stimulated with 50 ng/ml PMA for 15 min at 37° C.

### RNA Isolation

Mouse tissues were homogenized in Trizol (Invitrogen, Carlsbad Calif.) using an Ultraturrax (IKA, Staufen, Germany) disperser. The aqueous layer was mixed with an equal volume of 70% ethanol then RNA purified using an RNeasy kit (Qiagen, Valencia Calif.) according to the manufacturer's instructions. DNA digestion was performed using a Turbo-DNA-free kit (Ambion, Austin Tex.) following the manufacturer's protocols.

### Real Time RT-PCR

150 ng of RNA was converted to cDNA and amplified in one step using a Quanti-Tect SYBR Green RT-PCR kit (Qiagen). CD70 transcripts were quantitated by real-time RT-PCR using a Rotor-Gene 3000 (Corbett Research, Sydney, Australia) and previously published protocols (Gorelik, 2007; herein incorporated by reference in its entirety). The amplification conditions were: 30 min at 50° C., 15 min at 95° C., 40 cycles of 15s at 94° C., 20s at 56° C. and 30s at 72° C. followed by a final extension at 72° for 5 min. Transcript expression levels were normalized to GAPDH. The primers were: mouse GAPDH Fw: 5′-CAACGACCCCTTCATTGAC CTC-3′ (SEQ ID NO. 1); Rv: 5′-GCCTCACCCCATTT GATGTTAGTG-3′ (SEQ ID NO. 2), mouse CD70 Fw: 5′-TGGCTGTGG GCATCTG CTC-3′ (SEQ ID NO. 3); Rv:5′-ACATCTCCGTGGACCAGGTATG- (SEQ ID NO. 4), and mouse DNMT1 Fw: 5′-GGAAGGCTACCTGGCTAAAGTCAAG-3′ (SEQ ID NO. 5); Rv: 5′-ACTGAAAGGGTGTCACTGTCCGAC-3′ (SEQ ID NO. 6). The PCR products were fractionated on a 2% agarose gel and stained with ethidium bromide.

Product quality was determined by melting curves. A series of five dilutions of one RNA sample were included to generate a standard curve, and this was used to obtain relative concentrations of the transcript of interest in each of the RNA samples. In each experiment, water was included as a negative control. GAPDH amplification, as described above, was used to confirm that equal amounts of total RNA were added for each sample, and that the RNA was intact and equally amplifiable among all samples.

### DNA Isolation and PCR

Mice were screened for the presence of the dnPKCδ and CD2rtTA transgenes by PCR using genomic DNA isolated from tail-biopsy (Qiagen Blood & Tissue Kit). The PCR primers specific to each gene were obtained from Integrated DNA Technologies (IDT, Coralville, Iowa) and the sequences were as follows: dnPKCδ Fw: 5′-TTCAGTGATAGAGAACGTATG-3′ (SEQ ID NO. 7) and Rv; 5′-CAGCACAGAA AGGCTGGCTTGCTTC-3′(SEQ ID NO. 8). The primer sequences used for CD2rtTA were previously described. The protocol consisted of 40 cycles of incubation at 94° C. for 15 s, 55° C. for 20 s, and 72° C. for 30 s, followed by extension for 10 min at 72° C. A melting curve analysis was performed to demonstrate the specificity of the PCR product as a single peak.

### Protein Isolation

Following culture and stimulation, cells were centrifuged, resuspended in RIPA buffer (50 mM Tris-HCL, pH 7.4, 150 mM NaCl, 0.25% deoxycholic acid, 1% NP-40, 1 mM EDTA, 100 μg/ml PMSF, 100 μM sodium orthovanadate, 1 mM DTT and a protease inhibitor cocktail (Roche, Indianapolis Ind.), rotated at 4° C. for 30 min, insoluble material removed by centrifugation at 16000×g for 30 min and the supernatant saved as whole cell lysate. The protein was quantitated using the BCA Protein Assay (Pierce, Rockford, Ill.).

### Immunoblotting

20 μg of protein was diluted in Laemmli loading buffer and denatured by boiling for 5 min followed by electrophoresis in 10-12% SDS-polyacrylamide gels. The fractionated proteins were then electrophoretically transferred to nitrocellulose membranes (Schleicher and Schuell, Keene, N.H.) and stained with Ponceau S (Sigma) to verify equal loading. Membranes were blocked for 1 h in Tris-buffered saline (TBS) containing 0.1% Tween (Sigma) and 5% nonfat dry milk (Bio-Rad). After a 16-hour incubation with the kinase specific antibody in TBS, 0.1% Tween, 5% FBS, blots were washed three times with TBS containing 0.1% Tween and incubated with a horseradish peroxidase-linked secondary antibody for 1 h. After three washes with TBS-0.1% Tween, the membranes were treated using the ECL chemiluminescence detection system (Amersham, Piscataway, N.J.), exposed to X-Ray film (Kodak) and developed to visualize the labeled protein bands. Molecular mass was estimated by comparison of sample bands with prestained molecular mass markers (Bio-Rad, Hercules Calif.). For quantitative studies, the bands on X-ray films were scanned and analyzed with ImageQuant 5.2 software (Amersham). Where indicated, blots were stripped and reprobed with the corresponding antibody. Values were normalized with respect to β-actin and/or total kinase content as indicated.

### Anti-dsDNA Antibody Quantitation

Serum anti-dsDNA IgG antibodies were measured by ELISA. Microtiter plates (Costar, Corning, N.Y.) were coated overnight at 4° C. with 10 μg plasmid dsDNA, then 5 μL of mouse sera were added to each well in 100 μl of buffer and incubated overnight at 4° C. Bound anti-dsDNA antibody was detected by chemiluminescence using HRP-goat anti-mouse IgG (Bethyl Lab Inc) at 450 nm in a spectrophotometer equipped with Softmax Pro software (Molecular Devices, Sunnyvale, Calif.). Murine monoclonal anti-dsDNA antibody (Chemicon) was used for the standard curve.

### Histopathology

Double transgenic dnPKCδ/CD2rtTA mice were given 4 mg/ml dox/5% sucrose in their drinking water for 20 wks. Double transgenic animals treated with 5% sucrose were used as controls. At indicated times, their kidneys and lungs were removed. Kidneys were divided in two, one half embedded in O.C.T. (Thermo Fisher) and frozen in liquid nitrogen while the other half and the lung were fixed in 10% formalin, paraffin embedded and stained with hematoxylin and eosin. Five micron sections were cut from the frozen tissue and fixed for 10 minutes in ice cold acetone. 10% horse serum/PBS was used to block non-specific sites and the sections were stained with a 1:50 dilution of biotin-Goat anti-mouse IgG (Fc specific) antibody (US Biologicals)/FITC-Streptavidin (BD Pharmingen) to detect IgG depositions.

### Example 8

### Generation of dnPKCδ Double Transgenic Mice

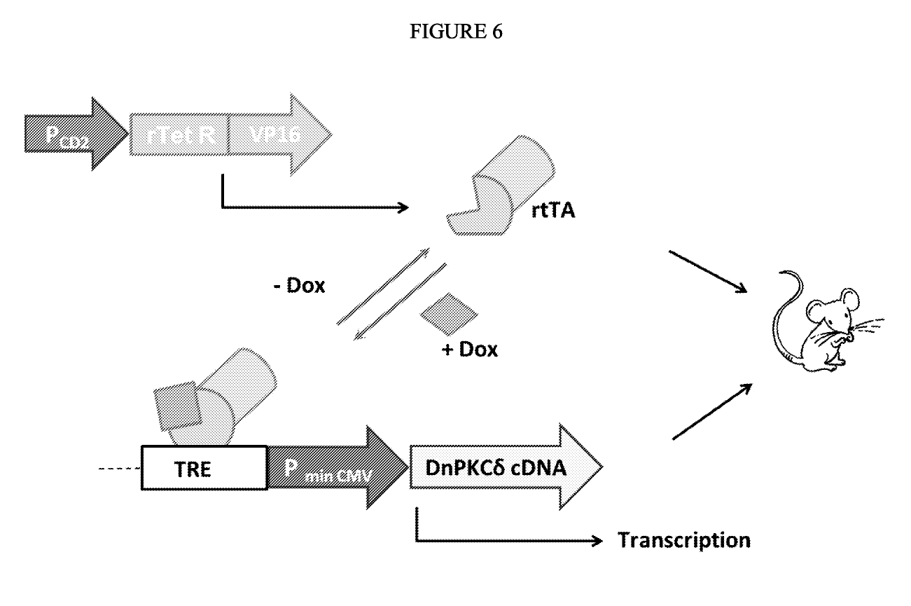

A dnPKCδ/CD2-rtTA double transgenic mouse was generated that expresses a dominant negative PKCδ (dnPKCδ) selectively in T cells only in the presence of dox as described above. A dnPKCδ, containing a K³⁷⁶R point mutation at the ATP binding site (Li, 1999; herein incorporated by reference in its entirety), was cloned into the pTRE2 vector containing a tetracycline response element, then the construct was injected into fertilized eggs from C57BL/6 X SJL mice and implanted into pseudopregnant females. Mice with the transgene were backcrossed onto an SJL background and bred with SJL transgenic strain containing the reverse tetracycline transactivator (rtTA) under the control of a CD2 promoter (CD2-rtTA). The rtTA only binds the tetracycline response element recognition sequence in the presence of dox. Administering dox in the drinking water induces expression of the dnPKCδ transgene specifically in T cells (FIG. 6).

### Example 9

### Leakiness and Inducibility

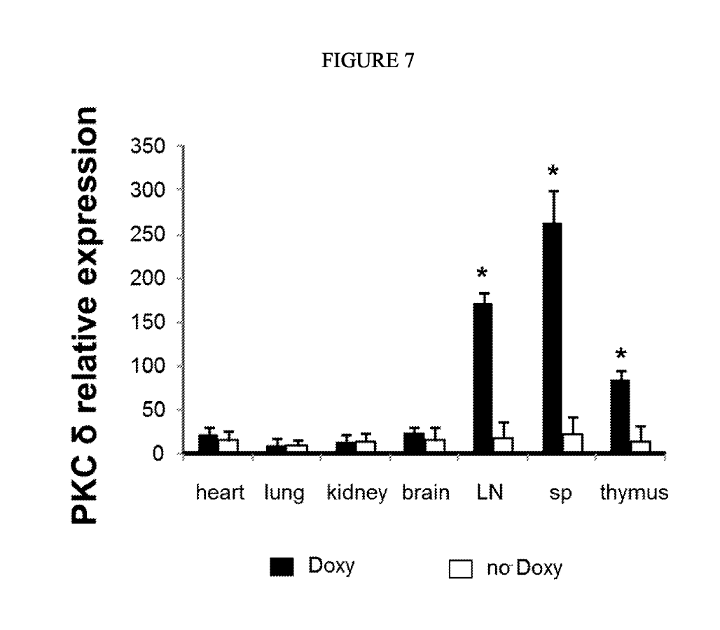

Mice were selected in which dnPKCδ expression was restricted to CD2 T cells and expressed only in the presence of dox. DnPKCδ/rtTA double transgenic mice were given dox/sucrose or sucrose alone in the drinking water for two weeks. The mice were then sacrificed and the expression of dnPKCδ mRNA was compared in tissues including the heart, lung, liver, brain, spleen, lymph nodes and thymus by RT-PCR. Mice in which the dnPKCδ was expressed only in lymph nodes, spleen and thymus, and only in the presence of dox (FIG. 7), were selected and bred.

### Example 10

### T Cell ERK Phosphorylation is Decreased in dnPKCδ/rtTA Mice Receiving Dox

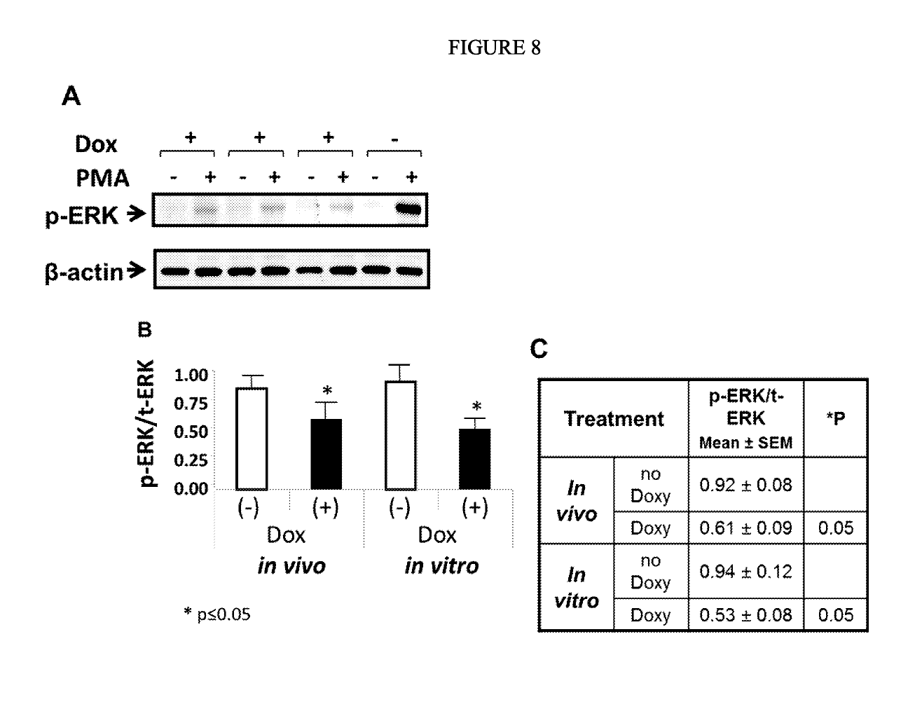

Experiments were conducted during development of embodiments of the present invention to determine if dnPKCδ/rtTA mice receiving dox had decreased ERK activation following PMA stimulation. CD3+ T cells were isolated from the spleen of dnPKCδ/CD2-rtTA mice receiving dox in vivo for two weeks, stimulated or not with PMA, then total and phosphorylated ERK were compared in to CD3+ T cells isolated from the spleens of control animals receiving water supplemented with sucrose but not dox. FIG. 8A shows a representative immunoblot comparing unstimulated and PMA stimulated ERK phosphorylation in T cells from 3 mice receiving dox to a control receiving just sucrose in the drinking water. Reduced ERK phosphorylation is seen in T cells from the dox-treated mice relative to controls following PMA stimulation (FIG. 8A). A similar decrease in PMA-stimulated ERK signaling was observed in CD3+ splenic T cells from 4 dnPKCδ/CD2-rtTA mice receiving dox to 4 receiving just sucrose in vivo, and in T cells from 5 mice cultured in vitro with or without dox for 24 h (FIG. 8B). These experiments demonstrate that T cells lacking PKCδ activity, express a reduced ERK signaling as was observed in lupus T cells.

### Example 11

### DnPKCδ Expression Decreases T Cell Dnmtl Levels and Increases Methylation Sensitive Gene Expression

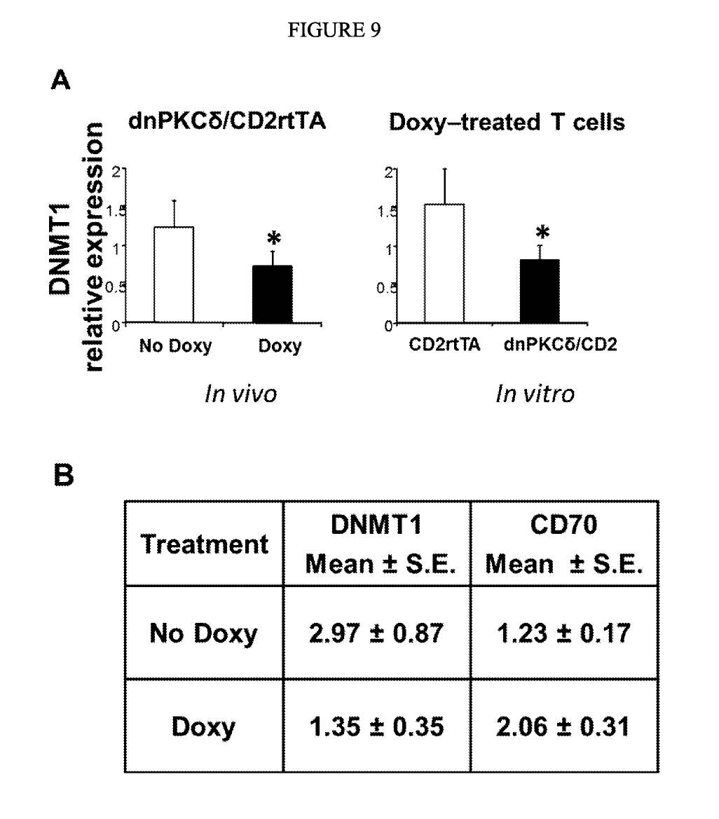

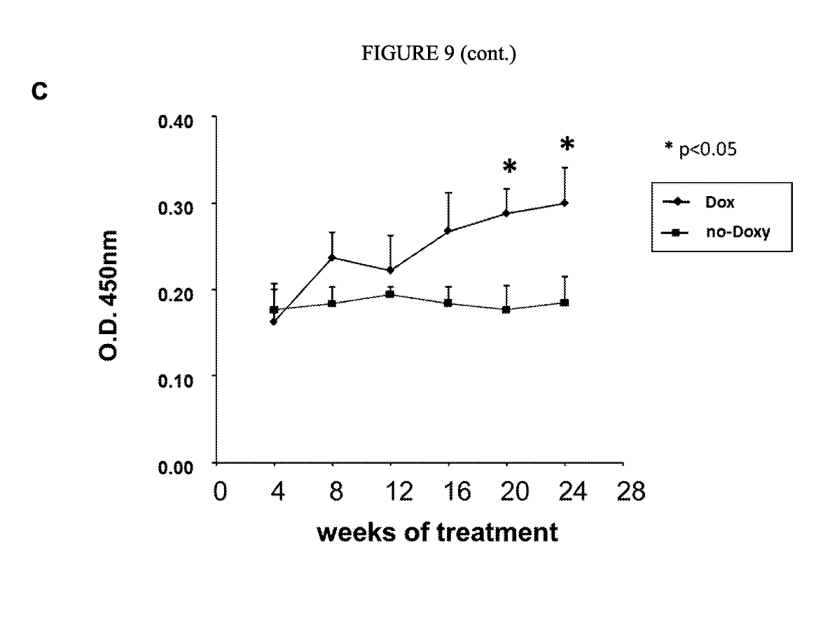

Experiments were conducted to determine whether decreased ERK pathway signaling in T cells from patients with active lupus contributes to the development of autoimmunity by decreasing Dnmt1 levels, resulting in DNA demethylation and overexpression of genes normally suppressed by DNA methylation. Double transgenic mice were given dox plus sucrose or sucrose alone for 2 weeks. The mice were then sacrificed, RNA isolated from their spleens, then Dnmt1 mRNA levels were measured by real time RT-PCR. An approximately 45% decrease in Dnmt1 transcripts was observed only in animals receiving with dox (FIG. 9A). Controls included comparing Dnmt1 levels in dox treated mice transgenic for just the CD2rtTA with dox treated dnPKCδ/CD2rtTA mice. Only double transgenic mice receiving dox decreased Dnmt1 levels (FIG. 9B), confirming that both transgenes and dox are required to decrease Dnmt1 levels in the T cells of these mice.

To determine the effects of the dnPKCδ on methylation sensitive T cell gene expression, mice were treated or not with dox for 2 weeks. Measurements were made of mRNA CD70 expression (Gorelik, 2007; herein incorporated by reference in its entirety). FIG. 9C compares CD70 mRNA expression in CD3+ T cells from the dnPKCδ/CD2-rtTA mice with and without dox. CD70 expression was significantly increased in T cells from dox-treated mice relative to controls (FIG. 9C).

### Example 12

### Inducing dnPKCδ Expression in T Cells is Sufficient to Cause Lupus-Like Autoimmunty

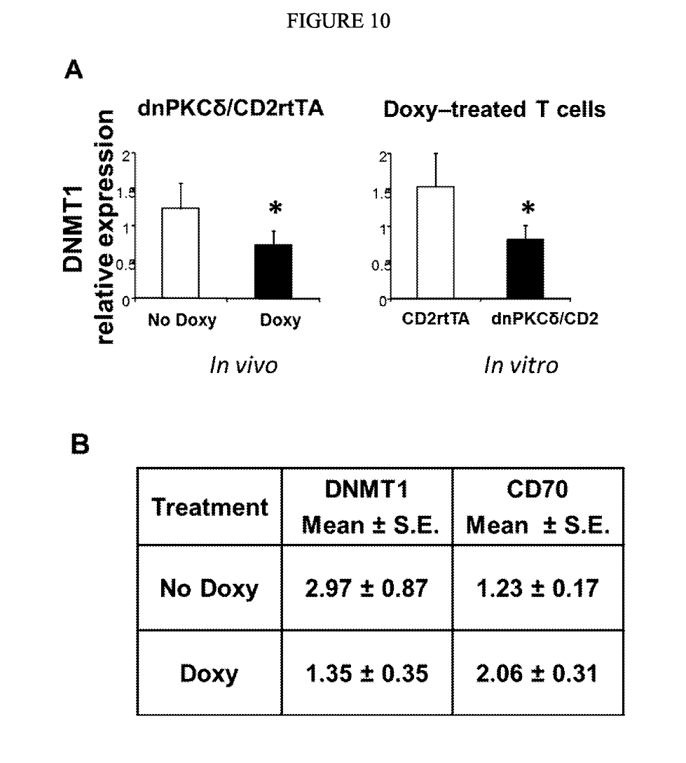

Experiments were conducted during the development of embodiments of the present invention to determine if the double transgenic develop anti-dsDNA antibodies when given dox. The double transgenic mice were given sucrose or sucrose plus dox in their drinking water, and IgG anti-dsDNA antibodies were measured serially over time by ELISA. FIG. 10 shows that mice receiving dox, but not controls, developed significantly higher levels of anti-dsDNA than controls animals.

### Example 13

### Glomerulonephritis and Histopathology in Double Transgenic Mice

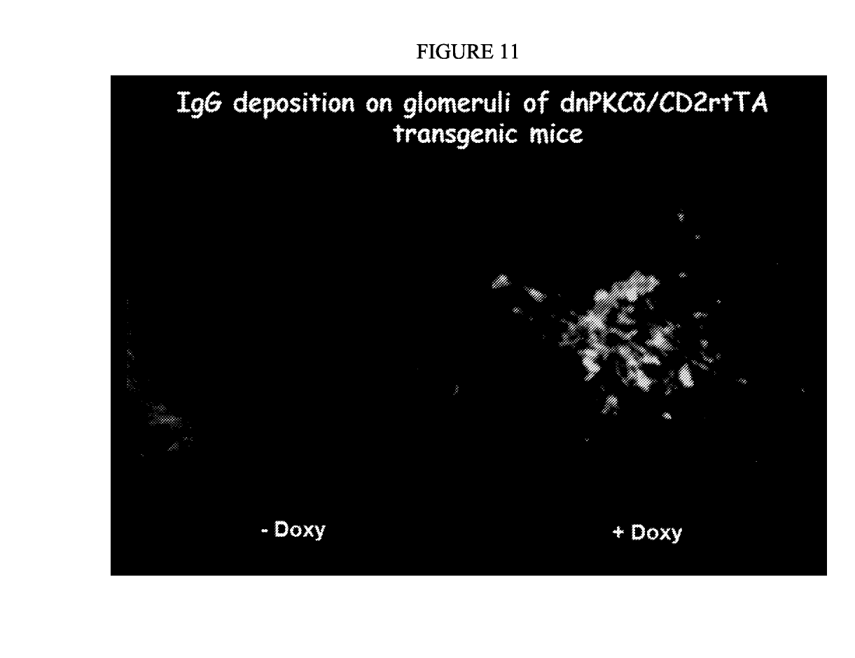

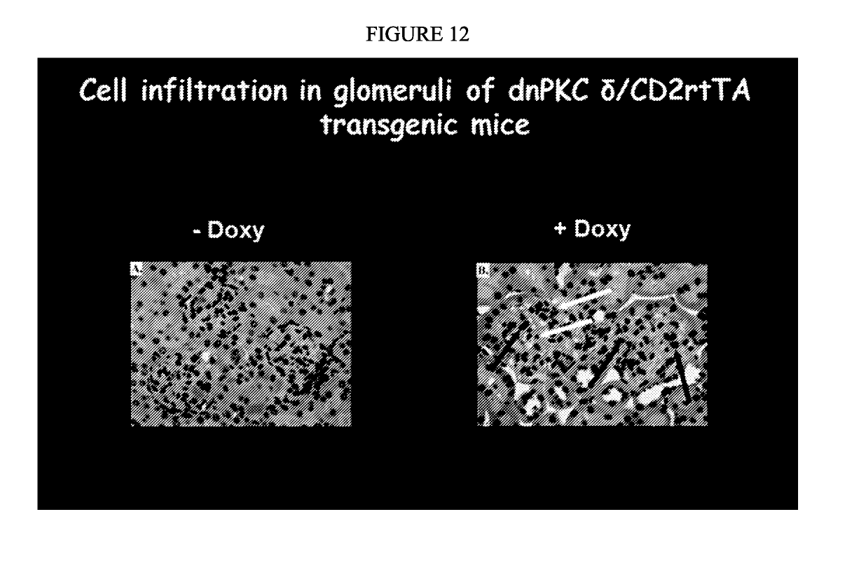

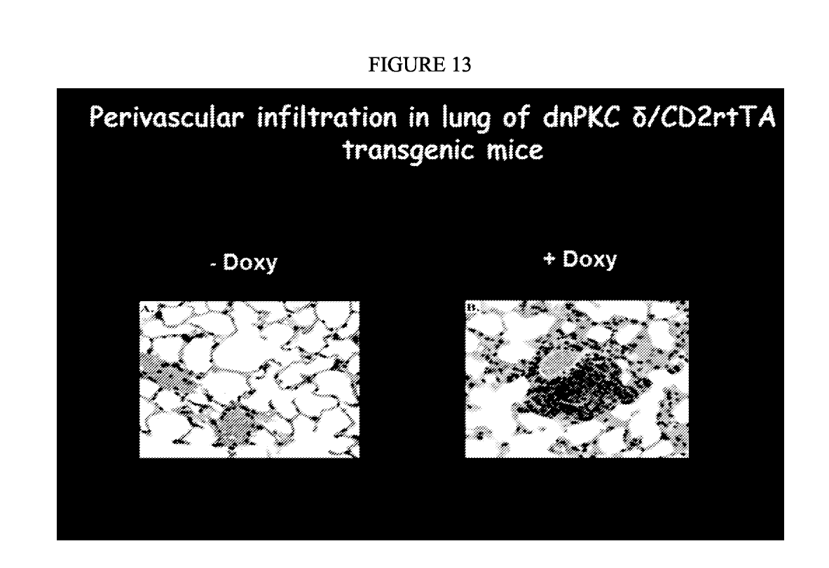

Experiments were conducted during the development of embodiments of the present invention to investigate the role of defective PKCδ on glomerulonephritis observed in lupus Kidneys from double transgenic dnPKCδ/CD2rtTA mice were removed from animals pretreated or not with Doxy and the histology shown evidence of glomerulonephritis in those Doxy-treated animals. Immunohistochemical staining noted IgG deposition along capillary walls and in the mesangial region of glomeruli from transgenic animals pretreated with Doxy (FIG. 11). It was also observed perivascular infiltration of leukocytes in glomeruli (FIG. 12), lungs (FIG. 13) and liver of transgenic animals pretreated with Doxy. None of these abnormalities were observed in the absence of Doxy or wild type animals.

Data, results, and a discussion relating to Examples 14-18 are published in Hewagama, J. Autoimmun. 2013 Feb. 19 pii: S0896-8411(12)00152-7; herein incorporated by reference in its entirety. The results obtained and conclusions drawn therefrom are specifically incorporated by reference.

### Example 14

### Material and Methods

### Examples 15-17

### Human Subjects

Healthy men and women ages 23-35, years were recruited by advertising from the general population at the University of Michigan (Ann Arbor, Mich.). Lupus patients were recruited from the Michigan Lupus Cohort and met patient's criteria for lupus (Tan Em, 1982; herein incorporated by reference in its entirety). The disease activity was estimated using Systemic Lupus Erythematosus Disease Activity Index (SLEDAI). Active disease was defined as a SLEDAI score≧4. For gene expression analysis by RT-qPCR, we used CD4+ cells from female lupus patients with an age range between 20 and 69 years, and male lupus patients with an age range between 18 and 74. For miRNA high throughput SBI profiling; active female lupus patients (Ages 27-57) and male lupus patients (Ages 23-40) with SLEDAIs≧4 were used. For methylation analysis, female lupus patients of ages between 24 and 61 were used. (Sawalha, 2012; Bombardier, 1992; herein incorporated by reference in their entireties).

### Cells, Cell Culture and Nucleic Acid Purification

For in vitro demethylation assay, isolated CD4+ cells were cultured with 5 μM 5-AzaC for 72 hours. The cells were then treated (restimulated) or not with 5 ng/ml PMA and 500 ng/ml ionomycin or not for an additional 6 hr, and CD4+ cells were recovered and washed 2 times with PBS. Total RNA including miRNAs was isolated using miRNeasy kit (Qiagen) for miRNA and mRNA expression analysis. Genomic DNA was isolated using Qiagen DNeasy Blood & Tissue Kit. (Hewagama, 2009; Lu, 2007; Quddus, 1993; herein incorporated by reference in their entireties).

### CXCR3, OGT and CBL Protein Quantitation

CD4 [symbol] T cell CXCR3 levels were measured by flow cytometry using antibodies from Santa Cruz Biotechnology. OGT was measured by solubilizing bead-purified CD4 [symbol] T cells, fractionating the lysates by electrophoresis, transferring the proteins to nitrocellulose membranes, then probing with anti-OGT antibodies (Abcam). CBL protein levels were measured by immunoblotting with anti-CBL antibody (BD Biosciences) and approaches similar to OGT. Protein bands were visualized using an ECL chemiluminescence detection system, then scanned and quantified with ImageQuant 5.2 software. Values were normalized to b-actin and levels expressed relative to non-stimulated CD4 [symbol] T cells. (Basu, 2009; Lu, 2007, Gorelik, 2012; herein incorporated by reference in their entireties).

### Transfection

Isolated CD4+ cells from healthy donors were cultured for 3 days in complete media and 20 U/ml IL2. Cells were then incubated in fresh media, without exogenous IL-2, for 24 hours prior to transient transfection with 100 nmol miRNA mimic (Qiagen) in the presensnce or absence of the 100 nmol anti-sense miRNA inhibitor (Qiagen) with the Amaxa Nucleofactor System (Lonza) according to manufacturer's instruction. Cells were harvested 30 hours after transfection, and target mRNAs were quantified by RT-qPCR.

### MicroArray

T cell RNA samples were analyzed on Affymetrix (Santa Clara, Calif.) GeneChip Human Genome Plus 2.0 (HG-133 Plus 2.0) microarrays by the University of Michigan Comprehensive Cancer Center (UMCCC) Affymetrix and Microarray Core Facility. The resulting data were then analyzed using the Genomatix ChipInspector program. Differential gene expression was detected using untreated as the control, and a false discovery rate (FDR)<4% was applied to identify differentially expressed genes. Microarray data mining was performed using BiblioSphere, Gene2Promoter and GEMS-Launcher applications of the Genomatix software suite; Gene Ontology (GO) classifications were performed using the BiblioSphere Biological Process filter.

Quantitative Real Time-PCR(RT-qPCR) for mRNA Expression Analysis

Up to 1000 ng total RNA from CD4+ T cells was transcribed into cDNA using Transcriptor First Strand cDNA synthesis kit (Roche) according to the manufacture's instruction. The cDNA was then subjected to real-time PCR using a Rotor-Gene 3000 thermocycler (Corbett). Roche (Basle) FastStart universal SYBR green master (Roche) mix containing 1 μl template cDNA and 0.5 μM forward and reverse primers in a total volume of 20 μl was annealed at 56° C. for a total of 40 cycles. The fold change of expression was calculated using ACTB and 18sRNA as internal reference genes.

### MicroRNA Expression Profiling

Genome-wide qPCR miRNA expression profiling was performed using (System Biosciences (SBI), Cat# RA660A-1) profiler. Briefly, 800 ng totals RNA containing miRNA was converted to complementary DNA with the QuantiMir kit, according to the manufacturer protocol. Real time PCR in 384 well format using miRNA-specific primers was performed using ABI 7900 real-time PCR system at University of Michigan microarray or sequencing core facilities. Resulting data was analyzed by using Human miRNome profiler software provided by SBI.

To identify methylation sensitive mRNAs, miRNA expression profiles were compared between 5-AzaC treated CD4+ cells and non-treated controls from healthy men and women. Differentially expressed miRNAs were identified. Then the X chromosome encoded miRNA's increased by 5-azaC in women, compared to men were identified. These miRNAs were compared with the list of X-linked miRNAs upregulated in women with lupus relative to men with lupus (women with active lupus vs. men with active lupus).

Target mRNA Prediction

Most likely targets for the miRNAs of interest were identified by Target Scan. Among the list of predicted miRNA targets; the top 100 with highest scores for the probability of conserved targeting and the context score were selected for functional analysis.

### Functional Analysis of the Target Genes

The target genes identified as above were subjected to further analysis using DAVID and ConceptGen program.

### Genomic Sequences and the Primer Design

Genome reference sequences flanking OGT, CXCR3 promoter regions and the miRNAs were extracted from (GRCh37/hg19) assembly of UCSC genome browser. The primers specific to the template sequences were obtained using NCBI Primer-BLAST tool.

### Methylated DNA Capture by Affinity Purification (MeCAP) and Quantitation of Enriched DNA

Genomic DNA from isolated CD4+ T cells (˜3×10⁶), was extracted using Qiagen DNeasy Blood & Tissue Kit. 3-5 μg isolated DNA was sonicated to average of ˜500 bp with a Covaris DNA shearing system at UM sequencing core. Fragments were size-evaluated using an Agilent 2100 Bioanalyzer. Approximately 400 ng sonicated DNA was subjected to MeCAP experiments using Active Motif METHYLCOLLECTOR Ultra Kit according to the manufacturer's instructions. DNA fragments from the methylation-enriched fraction were purified using QIAquick PCR Purification Kit (Qiagen). MeCAP enriched and reference input samples were quantitated using quantitative real time PCR(RT-qPCR) with primers designed for specific regions flanking the putative transcription start site of the gene promoters. Differentially methylated regions between patient and normal healthy controls were identified by comparing % enrichment (relative to input) of the 500 bp window of methylated fragments.

### Example 15

### X-Linked mRNA Encoding Genes Activated by DNA Methylation Inhibition in CD4+ T Cells from Women but not Men

Experiments were conducted during the development of embodiments of the present invention to identify X-linked mRNA encoding genes normally silenced by DNA methylation in T cells from women but not men. PBMC from healthy men and healthy women were stimulated with PHA, cultured with or without the irreversible Dnmt inhibitor 5-azaC for 72 hours, then stimulated or not with PMA and ionomycin. T cells were then purified, mRNA isolated and Affymetrix arrays used to identify X chromosome genes overexpressed in 5-azaC treated T cells from women relative to men. Using a false discovery rate≦4% and fold change≧1.2 female/male, four X chromosome genes were identified: CXCR3 (Xq13), OGT (Xq13), EDA (ectodysplasin A, Xq12-q13.1) and CD40LG (Xq26). All identified genes are distant from the pseudoautosomal regions at the ends of the X chromosome. EDA was overexpressed in unstimulated demethylated female cells, while CXCR3, OGT and CD40LG were overexpressed in restimulated, demethylated female cells. Ectodysplasin A is a membrane protein involved in cell-cell signaling during the development of ectodermal organs, and defects are a cause of anhidrotic ectodermal dysplasia, a disease occasionally but not strongly associated with immunodeficiency. CXCR3 encodes a chemokine receptor expressed on T cells and is implicated in T cell trafficking to the kidney in lupus nephritis. OGT encodes O-linked N-acetylglucosamine transferase, an enzyme that catalyzes the transfer of N-acetylglucosamine (GlcNAc) from UDP-N-acetylglucosamine to serines and threonines in cytoplasmic and nuclear proteins to form O-linked β-N-acetylglucosamine (O-GlcNAc), and serves as a signaling molecule. OGT has not been studied in lymphocytes or autoimmunity.

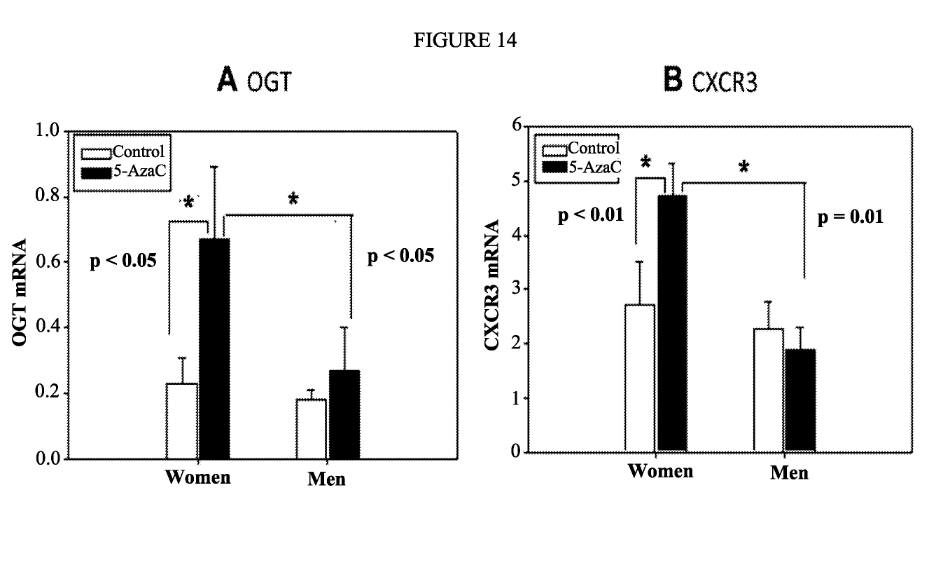

The sex-specific differences in gene expression were confirmed in experimentally demethylated CD4+ T cells from an additional 9 pairs of healthy men and women. Their PBMC were stimulated with PHA, treated with 5-azaC, and 72 hours later restimulated or not with PMA and ionomycin. CD4+ T cells were then purified using magnetic beads, RNA isolated, and OGT and CXCR3 mRNA levels quantitated by RT-PCR. FIG. 14A/B confirm that 5-azaC increases both CXCR3 and OGT transcripts more in CD4+ T cells from women than from men, consistent with the hypothesized increase in gene expression due to X chromosome demethylation in women but not men. (Lu, 2007; Enghard, 2009; Issad, 2010; Love, 2010; Golks, 2007; Mikkola, 2009; herein incorporated by reference in their entireties).

### Protein Confirmation.

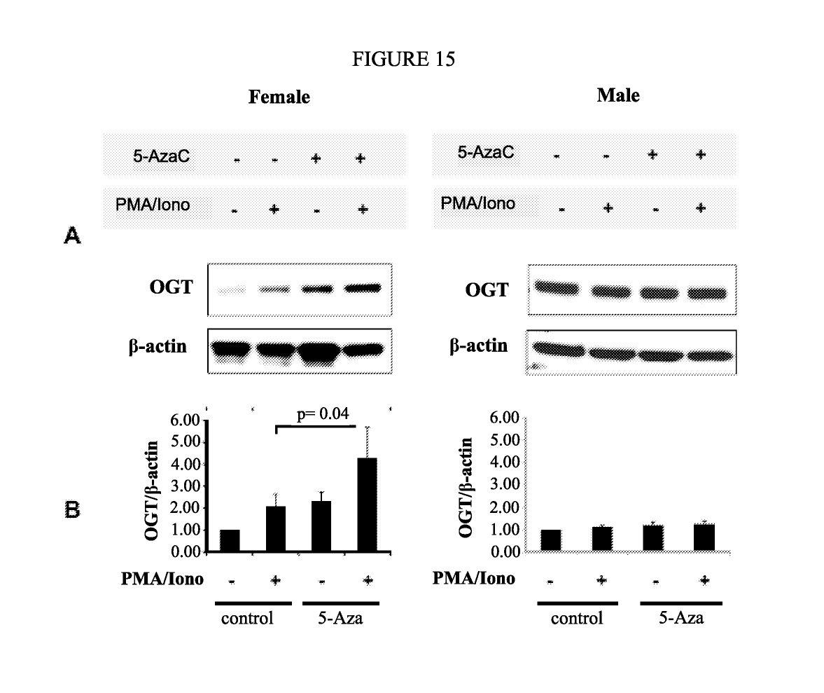

Experiments were conducted during the development of embodiments of the present invention to determine if 5-azaC also increased OGT and CXCR3 protein levels in CD4+ T cells. PBMC from N healthy men and women were stimulated with PHA, treated with 5-azaC, and 72 hours later restimulated with PMA and ionomycin as for an additional 6 hours as before. CD4+ T cells were isolated and OGT levels compared by immunoblotting. Experiments were conducted during development of embodiments of the present invention that demonstrate demethylated CD4+ T cells from women also express higher levels of OGT protein than men, consistent with the mRNA increase (FIG. 15).

CXCR3 protein levels were similarly compared on 5-azaC treated CD4+ T cells with and without restimulation using immunoblotting and flow cytometry. In contrast to OGT, using untreated and 5-azaC treated CD4+ T cells from male-female pairs, no increase in CXCR3 protein was observed (CXCR3MFI: female 320+91, male 316+108, mean±SEM) despite the increase in CXCR3 mRNA in the women. This suggests that additional post-transcriptional mechanisms regulate CXCR3 levels in CD4+ T cells, similar to those reported in NK cells (PMID: 12055219).

### DNA Methylation.

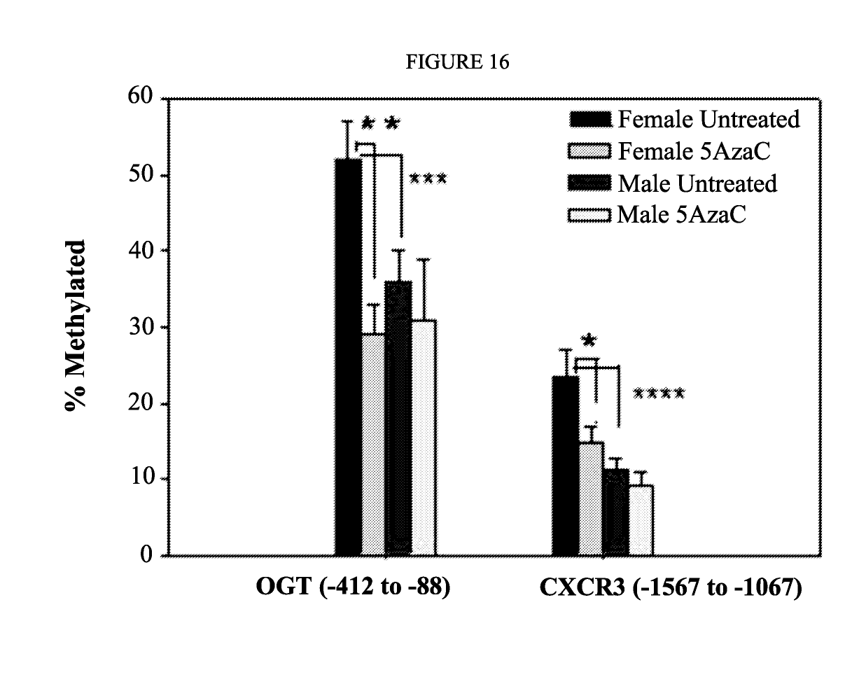

The overexpression of OGT and CXCR3 in 5-azaC treated CD4+ T cells from women relative to men is consistent with demethylation of regulatory regions on their silenced X chromosome. Experiments were conducted during the development of embodiments of the present invention to compare the methylation of putative regulatory regions for these genes in untreated and 5-azaC treated CD4+ T cells from men and women using a methylcytosine affinity purification (MeCAP) technique. DNA was purified from untreated and 5-azaC treated male and female CD4+ T cells, fragmented by sonication into approximately 500 bp fragments, methylated fragments affinity purified using recombinant methylcytosine binding proteins, and relative levels of the methylated fragments compared by PCR, using primers specific for specific regions flanking the putative transcription start site of the gene promoters. FIG. 16 shows that the region from −412 to −88 5′ to OGT transcription start site is significantly more methylated in women than in men, consistent with methylation of their inactive X, and that 5-azaC causes a significant demethylation of the same region in women but not men. Similarly, 5-azaC causes a significant decrease in methylation in a region located −1567 to −1067 5′ to the CXCR3 transcription start site, consistent with demethylation of sequences on their inactive X.

### Example 16

### Demethylation and Overexpression of X-Linked mRNA Genes in CD4+ T Cells from Women but not Men with Lupus

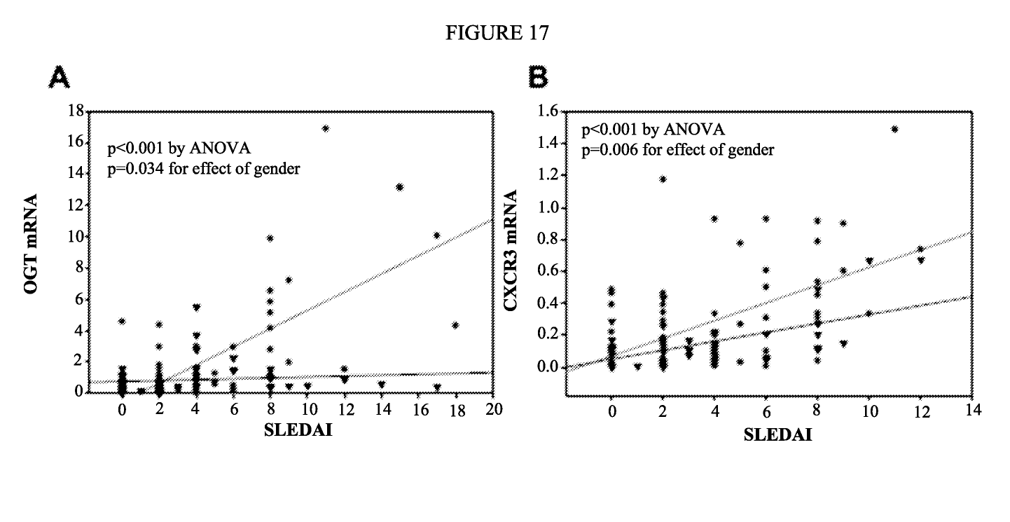

CD4+ T cells from women but not men with active lupus overexpress CD40LG, a B cell co-stimulatory molecule. Experiments were conducted during the development of embodiments of the present invention comparing OGT and CXCR3 mRNA and protein in men and women with lupus. FIG. 17A compares the levels of OGT mRNA relative to disease activity in CD4+ T cells from men and women with inactive and active lupus. While there is relatively little difference in OGT expression between men and women with relatively inactive disease, the women tend to express higher amounts with increasing disease activity, and overall the difference is highly significant. Similarly, FIG. 17B shows CXCR3 mRNA levels relative to disease activity in CD4+ T cells from men and women with inactive and active lupus. The women express higher amounts of CXCR3 mRNA. (Sawalha, 2012; Lu, 2007; herein incorporated by reference in their entireties).

### Protein Confirmation.

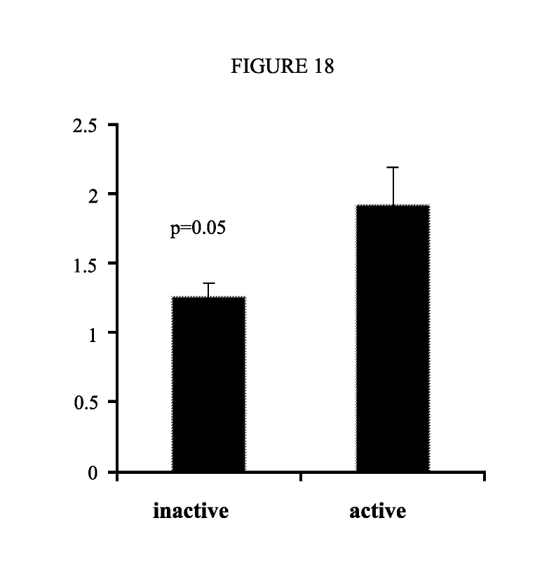

Experiments were conducted during the development of embodiments of the present invention to identify if a corresponding increase in OGT and CXCR3 protein was similarly sought in CD4+ T cells from women but not men with active lupus. FIG. 18 shows that women with active lupus overexpress OGT protein while men with active lupus do not as determined by immunoblotting. CXCR3 protein levels were compared by flow cytometry on CD4+ T cells from women and men with active lupus. However, similar to the results observed using experimentally demethylated T cells, there was no difference in the level of CXCR3 protein expression between the women and men (female:male MFI 1.07±1.08, mean±SD), again indicating that additional post-transcriptional mechanisms regulate CXCR3 levels in CD4+ T cells.

### DNA Methylation.

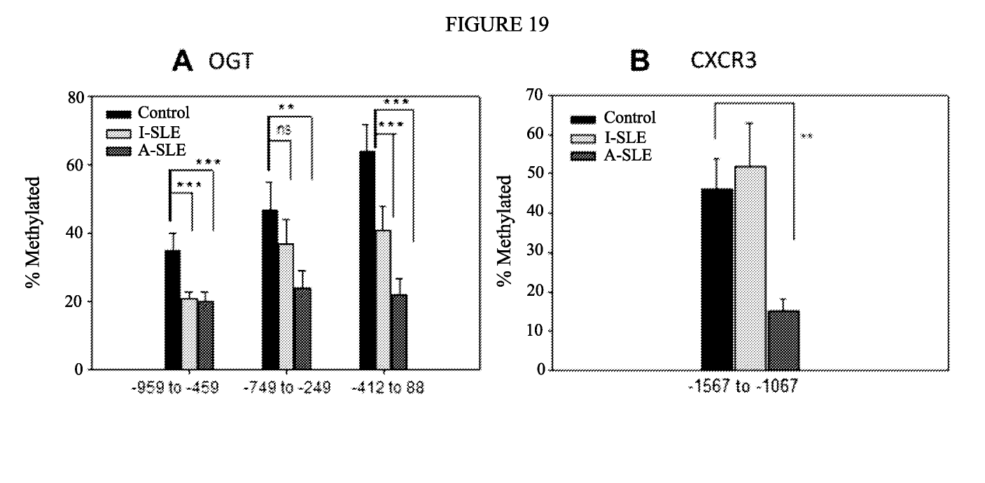

Experiments were conducted during the development of embodiments of the present invention to comparing methylation of the OGT and CXCR3 regulatory regions in CD4+ T cells from women with active lupus (SLEDAI 7.8±2.0, mean±SD), women with inactive lupus (SLEDAI 2.7±0.9, mean±SD) and healthy women, again using methylcytosine affinity purification. FIG. 19A shows that regions between −958 to +88 relative to the OGT transcription start site are methylated in normal women, but demethylate progressively with increasing disease activity in women with active lupus. FIG. 19B similarly shows that the region 5′ to the CXCR3 start site similarly demethylates in women with active lupus relative to women with inactive lupus and healthy controls, similar to the effects of 5-azaC and also consistent with demethylation of sequences on the inactive X.

### Example 17

### Identification of X-Linked miRNA's Silenced by DNA Methylation in Female T Cells

Experiments were conducted during the development of embodiments of the present invention to identify X-linked miRNA's increasing more in experimentally demethylated T cells from women than men and also overexpressed in women relative to men with active lupus. The human X chromosome encodes 128 miRNA while the Y chromosome encodes only 15 (Ensembl release 65—December 2011 © WTSI/EBI WTSI/EBI). PBMC from the peripheral blood of healthy men and healthy women ages 23-35 were stimulated with PHA and treated with 5-azaC as before. 72 hrs later CD4+ T cells were purified and miRNA's surveyed using PCR arrays detecting 80 X chromosome and 824 autosomal miRNAs but no Y miRNA's. Using a fold change≧1.5 and P≦0.05 in 5-azaC treated CD4 cells relative to untreated controls, 167 miRNAs were identified, 11 of which were encoded on the X chromosome and were significantly overexpressed in women but not men (Table 2), indicating of miRNA genes on the inactive X.

TABLE 2
X-linked miRNA's increased by 5-azaC in women but not men.

| | | | | | Female | | Male | |
|---|---|---|---|---|---|---|---|---|
| | | | | | (5AzaC/No 5Aza) | | (5AzaC/No 5Aza) | |
| | X chromosome MicroRNA | X-location | Distance between miRNAs in cluster | Chr. band | Fold change | p | Fold change | p |
| 1 | MIR-188(3p) | 49768109 | | p11.23 | 12.7 ± 3.0 | 0.03 | 4.1 ± 2.1 | 0.1 |
| 2 | MIR-98 | 53583184 | MIR98/LET 7F-2 = 969 bp | p11.22 | 1.7 ± 0.2 | 0.04 | 1.4 ± 0.2 | 0.06 |
| 3 | LET-7f-2 [symbol] | 53584153 | | p11.22 | 14 ± 5.7 | 0.03 | 4.7 ± 2.2 | 0.11 |
| 4 | LET-7f-2 | 53584153 | | p11.22 | 3.8 ± 1.2 | 0.02 | 0.5 ± 0.2 | 0.5 |
| 5 | MIR-421 | 73438212 | | q13.2 | 17.4 ± 5.0 | 0.01 | 7.8 ± 3.7 | 0.5 |
| 6 | MIR-106a | 1.34E+08 | | q26.2 | 5.2 ± 1.5 | 0.03 | 1.1 ± 0.4 | 0.08 |
| 7 | MIR-450b | 1.34E+08 | MIR450 cluster = 156 bp | q26.2 | 14.3 ± 4.4 | 0.03 | 3.8 ± 1.8 | 0.1 |
| 8 | MIR-450a | 1.34E+08 | | q26.2 | 2.4 ± 0.7 | 0.01 | 1.1 ± 0.8 | 0.1 |
| 9 | MIR-503 | 1.34E+08 | MIR 503/424 cluster = 286 bp | q26.2 | 14.3 ± 4.4 | 0.02 | 3.8 ± 1.8 | 0.1 |
| 10 | MIR-424 | 1.34E+08 | | q26.2 | 2.2 ± 0.5 | 0.01 | 1.1 ± 0.3 | 0.07 |
| 11 | MIR-508(3p) | 1.46E+08 | | q27.3 | 4.2 ± 1.1 | 0.03 | 1.1 ± 1.3 | 0.5 |

MIR in bold are overexpressed in both experimentally demethylated T cells from women relative to men and in T cells from women with active lupus relative to men with active lupus. [symbol]
indicates data missing or illegible when filed

Similar array studies were performed comparing miRNA's overexpressed in freshly isolated CD4 [symbol] T cells from women and men with active lupus. These studies identified a total of 18 X-linked miRNA's significantly overexpressed in women with active lupus relative to men with active lupus. These miRNA's are shown in Table 3. Of these 18 miR, 5 were also overexpressed in experimentally demethylated T cells from women relative to men (shown in bold in Table 3), suggesting that they may be silenced primarily by DNA methylation in women.

TABLE 3
X-linked miRNA's increased in women but not men with active

| MicroRNA | X chromosome coordinates | Chr. band | Relative expression [symbol] | p value |
|---|---|---|---|---|
| hsa-miR-532-3p | 49767754-49767844 [+] | p11.23 | 589.2 ± 471.2 | 3.1E−02 |
| hsa-miR-188-3p | 49768109-49768194 [+] | p11.23 | 563.3 ± 216.9 | 5.8E−04 |
| hsa-miR-188-5p | 49768109-49768194 [+] | p11.23 | 92.5 ± 36.7 | 2.3E−02 |
| hsa-miR-501-3p | 49774330-49774413 [+] | p11.23 | 1612.0 ± 592.24 | 1.2E−02 |
| hsa-miR-501-5p | 49774330-49774413 [+] | p11.23 | 234.8 ± 75.5 | 1.2E−02 |
| hsa-miR-502-5p | 49779206-49779291 [+] | p11.23 | 82.0 ± 36.63 | 3.9E−02 |
| hsa-miR-98 | [symbol] - [symbol] [−] | p11.22 | 9 ± 0.9 | 3.1E−02 |
| hsa-let-7f-2* | [symbol] - [symbol] [−] | p11.22 | 14.0 ± 4.8 | 3.1E−03 |
| hsa-miR-421 | [symbol] - [symbol] [−] | p13.2 | 139.9 ± 47.7 | 1.1E−03 |
| hsa-miR-361-5p | [symbol] - [symbol] [−] | q21.2 | 31.8 ± 16.53 | 1.8E−02 |
| hsa-miR-766 | 118780701-118780811 [−] | q24 | 225.8 ± 11.6 | 2.4E−02 |
| hsa-miR-450b-5p | 133674215-133674292 [−] | q26.3 | 43.6 ± 7.7 | 1.3E−03 |
| hsa-miR-503 | 133680358-133680428 [−] | q26.3 | 281.7 ± 123.4 | 8.4E−03 |
| hsa-miR-320d | [symbol] - [symbol] [−] | q27.1 | 106.4 ± 45.5 | 1.3E−02 |
| hsa-miR-507 | [symbol] - [symbol] [−] | q27.3 | 286.9 ± 157.1 | 1.6E−02 |
| hsa-miR-509-5p | [symbol] -146342143 [−] | q27.3 | 544.9 ± 278.5 | 1.2E−02 |
| hsa-miR-510 | 146353853- [symbol] [−] | q27.3 | 286.9 ± 157.1 | 2.7E−02 |
| hsa-miR-452 | 151128100- [symbol] [−] | q28 | 286.9 ± 157.1 | 3.6E−03 |

MIR in bold are overexpressed in both experimentally demethylated T cells from women relative to men and in T cells from women with active lupus relative to men with active lupus. [symbol]
indicates data missing or illegible when filed

### Example 18

### Functional Significance of Overexpressed miRNAs

The functional significance of overexpressed X-linked miRNAs was approached using DAVID (Database for Annotation, Visualization and Integrated Discovery) tools for functional clustering and ConceptGen tools for concept mapping. Target genes associated with deregulated T cell function were identified, then genes suppressing T cell activity, and finally selected miRNA's targeting these suppressors. Predicted targets for the 18 miRNA's overexpressed in women with lupus are shown in Table 4, and the 5 increased by 5-azaC and overexpressed in women with active lupus are shown in bold font.

TABLE 4
X-linked miRNA targets

| | c-CBL | CBA4 | PTEN | [symbol] | SOCS1 | SOCS2 | SOCS4 | SOCS5 | SOCS6 | SOCS7 |
|---|---|---|---|---|---|---|---|---|---|---|
| hsa-miR-532-3p | | | | | | | | | | |
| hsa-miR-188-3p | Y | | | | | | | | | Y |
| hsa-miR-188-5p | | | Y | | | | | | | |
| hsa-miR-501-3p | Y | | | | | | | | | |
| hsa-miR-501-5p | | | | | | | | | | |
| hsa-miR-502-5p | | Y | | | | | | | | |
| hsa-miR-98 | Y | | | Y | | | Y | | | Y |
| hsa-let-7f-2 [symbol] | Y | | | | | | | | | |
| hsa-miR-421 | Y | | | | | | | | | |
| hsa-miR-361-5 [symbol] | | | | Y | | | | | | |
| hsa-miR-766 | Y | | | | Y | | | | | |
| hsa-miR-450b-5p | | | | | | | | | Y | |
| hsa-miR-503 | | | | | | | | | Y | |
| hsa-miR-320d | | | Y | | | | | | | |
| hsa-miR-507 | | | | | | Y | | | | |
| hsa-miR-509-5p | | | | | | | | | | Y |
| hsa-miR-510 | | | | | | | | | | |
| hsa-miR-452 | | | | | | | | | | |

| | STATS8 | FAS | FASC | CASP3 | CASP7 | TNFRSF8 | TNFRSF18 |
|---|---|---|---|---|---|---|---|
| hsa-miR-532-3p | | | | | | | |
| hsa-miR-188-3p | | | | | | | |
| hsa-miR-188-5p | | | | | | | |
| hsa-miR-501-3p | Y | | | | | | |
| hsa-miR-501-5p | | | | | | | |
| hsa-miR-502-5p | | | | | | Y | |
| hsa-miR-98 | | Y | Y | Y | | | Y |
| hsa-let-7f-2 [symbol] | | | | | | | Y |
| hsa-miR-421 | Y | | | Y | | | |
| hsa-miR-361-5 [symbol] | | | | | | | |
| hsa-miR-766 | | | | | | | |
| hsa-miR-450b-5p | | | | | Y | | |
| hsa-miR-503 | | | | | | | |
| hsa-miR-320d | | | | | | | |
| hsa-miR-507 | Y | | | | Y | | |
| hsa-miR-509-5p | | | | | | | |
| hsa-miR-510 | | | | | | | |
| hsa-miR-452 | | | | | | | |

MIR in bold are overexpressed in both experimentally demethylated T cells from women relative to men and in T cells from women with active lupus relative to men withactive lupus. [symbol]
indicates data missing or illegible when filed

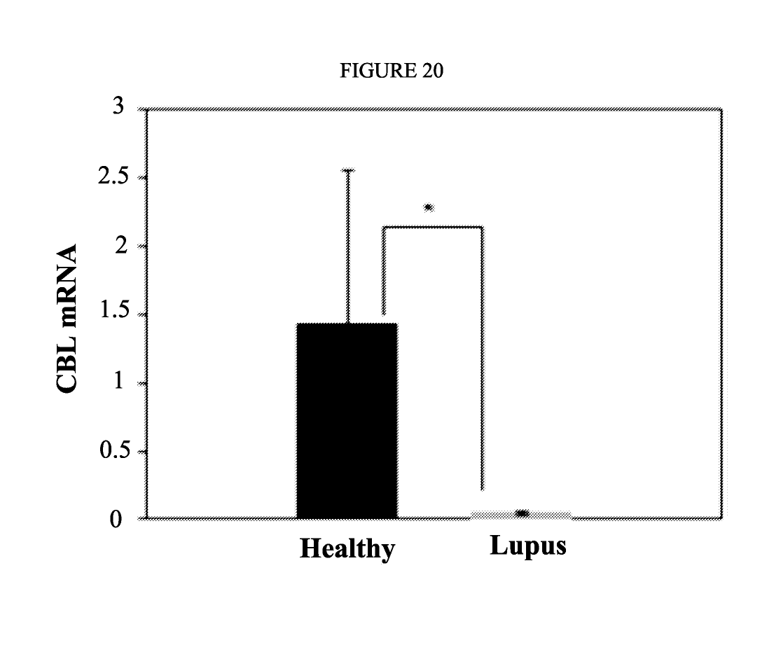

Experiments conducted during development of embodiments of the present invention identified CBL (c-CBL, E3 ubiquitin-protein ligase CBL) as targeted by the greatest number of X linked miRNA's overexpressed in experimentally demethylated T cells from women as well as T cells from women with lupus. CBL decreases T cell activation thresholds and co-stimulation requirements [Naramura, 2002; herein incorporated by reference in its entirety], at least in part by inhibiting ZAP-70 activation to lower the threshold for TCR activation and enhance signaling [Murphy, 1998; Pedraza-Alva, 2011; herein incorporated by reference in their entireties], and CBL protein levels are decreased in lupus T cells [Jury, 2004; herein incorporated by reference in its entirety]. FIG. 20 shows that CBL mRNA levels are significantly lower in CD4 [symbol] T cells from the women with lupus relative to healthy women, consistent with the decrease in CBL protein levels reported by others [Jury, 2004; herein incorporated by reference in its entirety]. However, there was no difference in CBL mRNA levels between the women with active and inactive lupus.

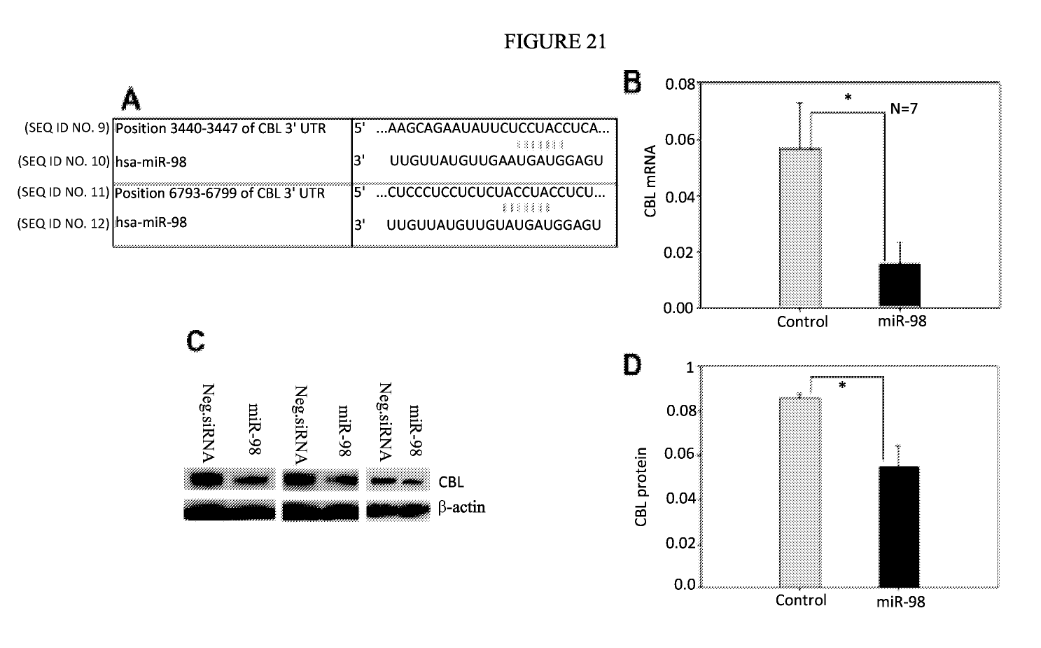

The target analyses also revealed that miR-98 was unique in being predicted to bind the largest number of suppressive transcripts (Table 4). Database analyses predicted that miR-98 would target two sites in the CBL (c-CBL, E3 ubiquitin-protein ligase CBL) 30 UTR (FIG. 21 A). Human CD4 [symbol] T cells were transfected with miR-98 or an anti-sense control in an expression construct, and CBL mRNA levels were compared in the transfected cells and untransfected controls. FIG. 21B shows that the miR-98 mimic, but not the antisense construct or empty vector, suppresses CBL mRNA expression. FIG. 21C shows an immunoblot demonstrating that miR-98 also decreases CBL protein but not b-actin, and FIG. 21D confirms that miR-98 decreases CBL protein.

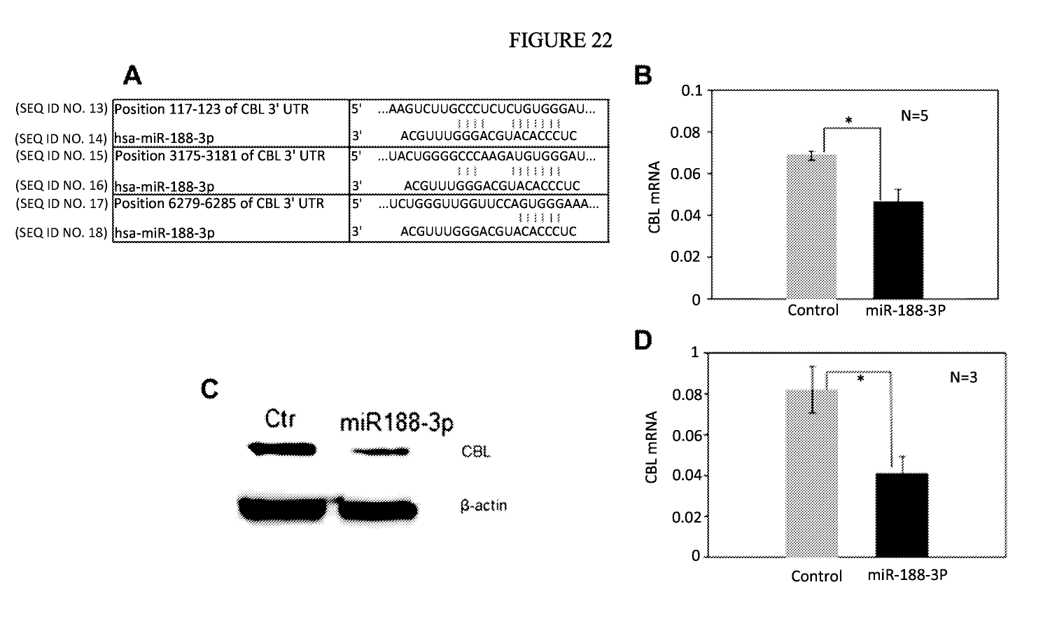

Table 3 also show that mir-188-3p was overexpressed in experimentally demethylated CD4 [symbol] T cells from women and in CD4 [symbol] T cells from women with active lupus like miR-98, and Table 4 shows that miR-188-3p would also be predicted to suppress CBL mRNA. Similar transfection studies confirmed that miR-188-3p also decreases CBL mRNA levels in T cells (FIG. 22).

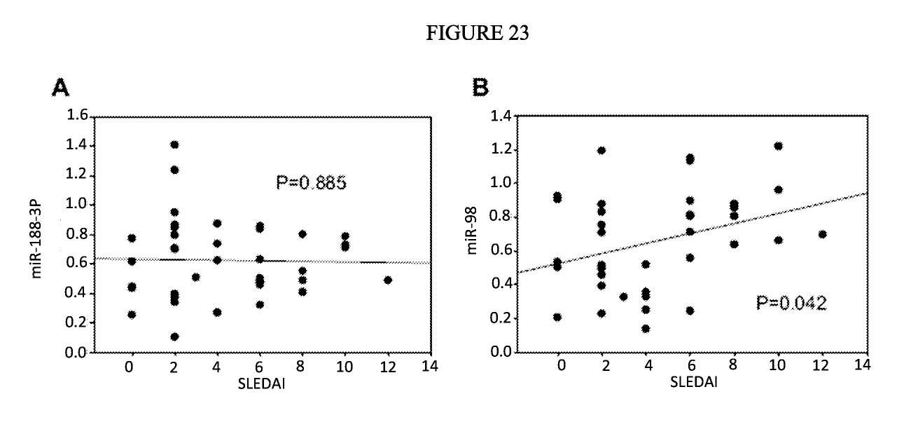

Since CBL mRNA is decreased in CD4 [symbol] T cells from women with both inactive and active lupus (FIG. 20), miR-98 and miR-188-3p levels were compared in CD4 [symbol] T cells from 20 women with inactive and active lupus. There was no change in miR-188-3p with respect to disease activity, indicating that miR-188-3p could help maintain the low CBL levels in T cells from women with inactive lupus. However, miR-98 levels increased slightly with increasing disease activity (FIG. 23).

Methylation of potential regulatory regions 5′ to the miR-98/Let 7-f2 gene cluster was compared in experimentally demethylated CD4 [symbol] T cells from women relative to men, and in CD4 [symbol] T cells from women with active lupus. FIG. 24A shows that this region significantly demethylates in 5-azaC treated T cells from women but not men, while FIG. 24B shows that the same region is demethylated in CD4 [symbol] T cells from women with both inactive and active lupus, with a further decrease in women with active relative to inactive lupus. Demethylation of this region in women with both inactive and active lupus is consistent with the increased miR-98 levels observed in women with both inactive and active lupus, and resembles the demethylation previously reported by our group in CD70, which is similarly demethylated in women with lupus independent of disease activity (Lu, 2005; herein incorporated by reference in its entirety).

All publications and patents mentioned in the present application and/or listed below are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

### REFERENCES

The following references, referred to throughout the specification by number, are herein incorporated by reference in their entireties.
Hewagama A, Richardson B. The genetics and epigenetics of autoimmune diseases. J Autoimmun 2009; 33(1):3-11.
Attwood J T, Yung R L, Richardson B C. DNA methylation and the regulation of gene transcription. Cell Mol Life Sci 2002; 59(2):241-57.
Bestor T, Laudano A, Mattaliano R, Ingram V. Cloning and sequencing of a cDNA encoding DNA methyltransferase of mouse cells. The carboxyl-terminal domain of the mammalian enzymes is related to bacterial restriction methyltransferases. J Mol Biol 1988; 203(4):971-83.
Deng C, Yang J, Scott J, Hanash S, Richardson B C. Role of the ras-MAPK signaling pathway in the DNA methyltransferase response to DNA hypomethylation. Biol Chem 1998; 379(8-9):1113-20.
MacLeod A R, Rouleau J, Szyf M. Regulation of DNA methylation by the Ras signaling pathway. J Biol Chem 1995; 270(19):11327-37.
Rouleau J, MacLeod A R, Szyf M. Regulation of the DNA methyltransferase by the Ras-AP-1 signaling pathway. J Biol Chem 1995; 270(4):1595-601.
Deng C, Lu Q, Zhang Z, Rao T, Attwood J, Yung R, et al. Hydralazine may induce autoimmunity by inhibiting extracellular signal-regulated kinase pathway signaling. Arthritis Rheum 2003; 48(3):746-56.
Yung R L, Quddus J, Chrisp C E, Johnson K J, Richardson B C. Mechanism of druginduced lupus. I. Cloned Th2 cells modified with DNA methylation inhibitors in vitro cause autoimmunity in vivo. J Immunol 1995; 154(6):3025-35.
Sawalha A H, Jeffries M, Webb R, Lu Q, Gorelik G, Ray D, et al. Defective T-cell ERK signaling induces interferon-regulated gene expression and overexpression of methylationsensitive genes similar to lupus patients. Genes Immun 2008; 9(4):368-78.
Deng C, Kaplan M J, Yang J, Ray D, Zhang Z, McCune W J, et al. Decreased Rasmitogen-activated protein kinase signaling may cause DNA hypomethylation in T lymphocytes from lupus patients. Arthritis Rheum 2001; 44(2):397-407.
Lu Q, Wu A, Richardson BC. Demethylation of the Same Promoter Sequence Increases CD70 Expression in Lupus T Cells and T Cells Treated with Lupus-Inducing Drugs. J Immunol 2005; 174(10):6212-6219.
Gorelik G, Fang J Y, Wu A, Sawalha A H, Richardson B. Impaired T cell protein kinase C delta activation decreases ERK pathway signaling in idiopathic and hydralazine-induced lupus. Immunol 2007; 179(8):5553-63.
Amos S, Martin P M, Polar G A, Parsons S J, Hussaini I M. Phorbol 12-myristate 13-acetate induces epidermal growth factor receptor transactivation via protein kinase Cdelta/c-Src pathways in glioblastoma cells. J Biol Chem 2005; 280(9):7729-38.
Basu A, Tu H. Activation of ERK during DNA damage-induced apoptosis involves protein kinase Cdelta. Biochem Biophys Res Commun 2005; 334(4):1068-73.
Kuriyama M, Taniguchi T, Shirai Y, Sasaki A, Yoshimura A, Saito N. Activation and translocation of PKCdelta is necessary for VEGF-induced ERK activation through KDR in HEK293T cells. Biochem Biophys Res Commun 2004; 325(3):843-51.
Zhang Q, Ye D Q, Chen G P, Zheng Y. Oxidative protein damage and antioxidant status in systemic lupus erythematosus. Clin Exp Dermatol 2010; 35(3):287-94.
Mansour R B, Lassoued S, Gargouri B, El Gaid A, Attia H, Fakhfakh F. Increased levels of autoantibodies against catalase and superoxide dismutase associated with oxidative stress in patients with rheumatoid arthritis and systemic lupus erythematosus. Scand J Rheumatol 2008; 37(2):103-8.
Kurien B T, Scofield R H. Free radical mediated peroxidative damage in systemic lupus erythematosus. Life Sci 2003; 73(13):1655-66.
Ahmad R, Rasheed Z, Ahsan H. Biochemical and cellular toxicology of peroxynitrite: implications in cell death and autoimmune phenomenon. Immunopharmacol Immunotoxicol 2009; 31(3):388-96.
Pacher P, Beckman J S, Liaudet L. Nitric oxide and peroxynitrite in health and disease. Physiol Rev 2007; 87(1):315-424.
Tan E M, Cohen A S, Fries J F, Masi A T, McShane D J, Rothfield N F, et al. The 1982 revised criteria for the classification of systemic lupus erythematosus. Arthritis Rheum 1982; 25(11):1271-7.
Bombardier C, Gladman D D, Urowitz M B, Caron D, Chang C H. Derivation of the SLEDAI. A disease activity index for lupus patients. The Committee on Prognosis Studies in SLE. Arthritis Rheum 1992; 35(6):630-40.
Katsiari C G, Kyttaris V C, Juang Y T, Tsokos G C. Protein phosphatase 2A is a negative regulator of IL-2 production in patients with systemic lupus erythematosus. J Clin Invest 2005; 115(11):3193-204.
Le Good J A, Ziegler W H, Parekh D B, Alessi D R, Cohen P, Parker P J. Protein Kinase C Isotypes Controlled by Phosphoinositide 3-Kinase Through the Protein Kinase PDK1. Science 1998; 281(5385):2042-2045.
Casamayor A, Morrice N A, Alessi D R. Phosphorylation of Ser-241 is essential for the activity of 3-phosphoinositide-dependent protein kinase-1: identification of five sites of phosphorylation in vivo. Biochem J 1999; 342 (Pt 2):287-92.
Knight Z A. For a PDK1 inhibitor, the substrate matters. Biochem J 2011; 433(2):e1-2.
Morgan P E, Sturgess A D, Davies M J. Increased levels of serum protein oxidation and correlation with disease activity in systemic lupus erythematosus. Arthritis Rheum 2005; 52(7):2069-79.
Steinberg S F. Distinctive activation mechanisms and functions for protein kinase Cdelta. Biochem. J. 2004; 384(3):449-459.
Kikkawa U, Matsuzaki H, Yamamoto T. Protein kinase C delta (PKC delta): activation mechanisms and functions. J Biochem 2002; 132(6):831-9.
Wu F, Wilson J X. Peroxynitrite-dependent activation of protein phosphatase type 2A mediates microvascular endothelial barrier dysfunction. Cardiovasc Res 2009; 81(1):38-45.
Srivastava J, Goris J, Dilworth S M, Parker PJ. Dephosphorylation of PKCdelta by protein phosphatase 2Ac and its inhibition by nucleotides. FEBS Lett 2002; 516(1-3):265-9.
Myles T, Schmidt K, Evans D R, Cron P, Hemmings B A. Active-site mutations impairing the catalytic function of the catalytic subunit of human protein phosphatase 2A permit baculovirus-mediated overexpression in insect cells. Biochem J 2001; 357(Pt 1):225-32.
Wang G, Pierangeli S S, Papalardo E, Ansari G A, Khan M F. Markers of oxidative and nitrosative stress in systemic lupus erythematosus: Correlation with disease activity. Arthritis Rheum 2010; 62(7):2064-2072.
Avalos I, Chung C P, Oeser A, Milne G L, Morrow J D, Gebretsadik T, et al. Oxidative stress in systemic lupus erythematosus: relationship to disease activity and symptoms. Lupus 2007; 16(3):195-200.
Nagy G, Koncz A, Perl A. T- and B-cell abnormalities in systemic lupus erythematosus. Crit. Rev Immunol 2005; 25(2):123-40.
Khan F, Siddiqui A A, Ali R. Measurement and significance of 3-nitrotyrosine in systemic lupus erythematosus. Scand J Immuno12006; 64(5):507-14.
Nishizuka Y. Protein kinase C and lipid signaling for sustained cellular responses. FASEB J 1995; 9(7):484-96.
Steinberg SF. Structural Basis of Protein Kinase C Isoform Function. Physiological Reviews 2008; 88(4):1341-1378.
Nagy G, Koncz A, Telarico T, Fernandez D, Ersek B, Buzas E, et al. Central role of nitric oxide in the pathogenesis of rheumatoid arthritis and systemic lupus erythematosus. Arthritis Res Ther 2010; 12(3):210.
Oates J C, Christensen E F, Reilly C M, Self S E, Gilkeson G S. Prospective measure of serum 3-nitrotyrosine levels in systemic lupus erythematosus: correlation with disease activity. Proc Assoc Am Physicians 1999; 111(6):611-21.
Rybin V O, Guo J, Sabri A, Elouardighi H, Schaefer E, Steinberg SF. Stimulus-specific differences in protein kinase C delta localization and activation mechanisms in cardiomyocytes. J Biol Chem 2004; 279(18):19350-61.
Denning M F, Dlugosz A A, Threadgill D W, Magnuson T, Yuspa S H. Activation of the Epidermal Growth Factor Receptor Signal Transduction Pathway Stimulates Tyrosine Phosphorylation of Protein Kinase C [IMAGE]. J. Biol. Chem. 1996; 271(10):5325-5331.
Konishi H, Yamauchi E, Taniguchi H, Yamamoto T, Matsuzaki H, Takemura Y, et al. Phosphorylation sites of protein kinase C Î′ in H2O2-treated cells and its activation by tyrosine kinase invitro. Proc Natl Acad Sci USA 2001; 98(12):6587-6592.
Kronfeld I, Kazimirsky G, Lorenzo P S, Garfield S H, Blumberg P M, Brodie C. Phosphorylation of protein kinase Cdelta on distinct tyrosine residues regulates specific cellular functions. Biol Chem 2000; 275(45):35491-8.
Kiroycheva M, Ahmed F, Anthony G M, Szabo C, Southan G J, Bank N. Mitogenactivated protein kinase phosphorylation in kidneys of beta(s) sickle cell mice. J Am Soc Nephrol 2000; 11(6):1026-32.
Wayne J, Sielski J, Rizvi A, Georges K, Hutter D. ERK regulation upon contact inhibition in fibroblasts. Mol Cell Biochem 2006; 286(1-2):181-9.
Khan F, Ali R. Antibodies against nitric oxide damaged poly L-tyrosine and 3-nitrotyrosine levels in systemic lupus erythematosus. J Biochem Mol Biol 2006; 39(2):189-96.
Webster R P, Roberts V H, Myatt L. Protein nitration in placenta—functional significance. Placenta 2008; 29(12):985-94.
Hedrich C M, Tsokos G C. Epigenetic mechanisms in systemic lupus erythematosus and other autoimmune diseases. Trends Mol Med 2011; 17(12):714-24.
Cooper G S, Stroehla B C. The epidemiology of autoimmune diseases. Autoimmun Rev 2003; 2:119e25.
Buyon J P, Petri M A, Kim M Y, Kalunian K C, Grossman J, Hahn B H, et al. The effect of combined estrogen and progesterone hormone replacement therapy on disease activity in systemic lupus erythematosus: a randomized trial. Ann Intern Med 2005; 142:953e62.
Huang J L, Yao T C, See L C. Prevalence of pediatric systemic lupus erythematosus and juvenile chronic arthritis in a Chinese population: a nation-wide prospective population-based study in Taiwan. Clin Exp Rheumatol 2004; 22:776e80.
Boddaert J, Huong du L T, Amoura Z, Wechsler B, Godeau P, Piette J C. Late-onset systemic lupus erythematosus: a personal series of 47 patients and pooled analysis of 714 cases in the literature. Medicine (Baltimore) 2004; 83:348e59.
Lockshin M D, Buyon J P. Estrogens and lupus: bubbling cauldron or another overrated Witches' Brew? Arthritis Rheum 2007; 56:1048e50.
Lockshin M D. Sex ratio and rheumatic disease: excerpts from an institute of medicine report. Lupus 2002; 11:662e6.
Scofield R H, Bruner G R, Namjou B, Kimberly R P, Ramsey-Goldman R, Petri M, et al. Klinefelter's syndrome (47, XXY) in male systemic lupus erythematosus patients: support for the notion of a gene-dose effect from the X chromosome. Arthritis Rheum 2008; 58:2511e7.
Cooney C M, Bruner G R, Aberle T, Namjou-Khales B, Myers L K, Feo L, et al. 46, X, del(X)(q13) Turner's syndrome women with systemic lupus erythematosus in a pedigree multiplex for SLE. Genes Immun 2009; 10:478e81.
Richardson B. Primer: epigenetics of autoimmunity. Nat Clin Pract Rheumatol 2007; 3:521e7.
Cornacchia E, Golbus J, Maybaum J, Strahler J, Hanash S, Richardson B. Hydralazine and procainamide inhibit T cell DNA methylation and induce autoreactivity. J Immunol 1988; 140:2197e200.
Basu D, Liu Y, Wu A, Yarlagadda S, Gorelik G J, Kaplan M J, et al. Stimulatory and inhibitory killer Ig-like receptor molecules are expressed and functional on lupus T cells. J Immunol 2009; 183:3481e7.
Quddus J, Johnson K J, Gavalchin J, Amento E P, Chrisp C E, Yung R L, et al. Treating activated CD4 [symbol] T cells with either of two distinct DNA methyltransferase inhibitors, 5-azacytidine or procainamide, is sufficient to cause a lupus-like disease in syngeneic mice. J Clin Invest 1993; 92:38e53.
Javierre B M, Richardson B. A new epigenetic challenge: systemic lupus erythematosus. Adv Exp Med Biol 2011; 711:117e36.
Lu Q, Wu A, Tesmer L, Ray D, Yousif N, Richardson B. Demethylation of CD40LG on the inactive X in T cells from women with lupus. J Immunol 2007; 179:6352e8.
Tan E M, Cohen A S, Fries J F, Masi A T, McShane D J, Rothfield N F, et al. The 1982 revised criteria for the classification of systemic lupus erythematosus. Arthritis Rheum 1982; 25:1271e7.
Sawalha A H, Wang L, Nadig A, Somers E C, McCune W J, Hughes T, et al. Sex-specific differences in the relationship between genetic susceptibility, T cell DNA demethylation and lupus flare severity. J Autoimmun 2012; 38:J216e22.
Bombardier C, Gladman D D, Urowitz M B, Caron D, Chang C H. Derivation of the SLEDAI. A disease activity index for lupus patients. The committee on prognosis studies in SLE. Arthritis Rheum 1992; 35:630e40.
Hewagama A, Patel D, Yarlagadda S, Strickland F M, Richardson B C. Stronger inflammatory/cytotoxic T-cell response in women identified by microarray analysis. Genes Immun 2009; 10:509e16.
Gorelik G J, Yarlagadda S, Richardson B C. PKCdelta oxidation contributes to erk inactivation in lupus t cells. Arthritis Rheum 2012.
Gorelik G, Fang J Y, Wu A, Sawalha A H, Richardson B. Impaired T cell protein kinase C delta activation decreases ERK pathway signaling in idiopathic and hydralazine-induced lupus. Immunol 2007; 179:5553e63.
Enghard P, Humrich J Y, Rudolph B, Rosenberger S, Biesen R, Kuhn A, et al. CXCR3 [symbol] CD4 [symbol] T cells are enriched in inflamed kidneys and urine and provide a new biomarker for acute nephritis flares in systemic lupus erythematosus patients. Arthritis Rheum 2009; 60:199e206.
Issad T, Masson E, Pagesy P. O-GlcNAc modification, insulin signaling and diabetic complications. Diabetes Metab 2010; 36:423e35.
Love D C, Krause M W, Hanover J A. O-GlcNAc cycling: emerging roles in development and epigenetics. Semin Cell Dev Biol 2010; 21:646e54.
Golks A, Tran T T, Goetschy J F, Guerini D. Requirement for O-linked N-acetylglucosaminyltransferase in lymphocytes activation. EMBO J. 2007; 26:4368e79.
Mikkola M L. Molecular aspects of hypohidrotic ectodermal dysplasia. Am J Med Genet A 2009; 149A:2031e6.
Hodge D L, Schill W B, Wang J M, Blanca I, Reynolds D A, Ortaldo J R, et al. IL-2 and IL-12 alter NK cell responsiveness to IFN-gamma-inducible protein 10 by down-regulating CXCR3 expression. J Immunol 2002; 168:6090e8.
Naramura M, Jang I K, Kole H, Huang F, Haines D, Gu H. c-Cbl and Cblb regulate T cell responsiveness by promoting ligand-induced TCR downmodulation. Nat Immunol 2002; 3:1192e9.
Murphy M A, Schnall R G, Venter D J, Barnett L, Bertoncello I, Thien C B, et al. Tissue hyperplasia and enhanced T-cell signalling via ZAP-70 in c-Cbl-deficient mice. Mol Cell Biol 1998; 18:4872e82.
Pedraza-Alva G, Merida L B, del Rio R, Fierro N A, Cruz-Munoz M E, Olivares N, et al. CD43 regulates the threshold for T cell activation by targeting Cbl functions. IUBMB Life 2011; 63:940e8.
Jury E C, Kabouridis P S, Flores-Borja F, Mageed R A, Isenberg D A. Altered lipid raft-associated signaling and ganglioside expression in T lymphocytes from patients with systemic lupus erythematosus. J Clin Invest 2004; 113: 1176e87.
Lu Q, Wu A, Richardson B C. Demethylation of the same promoter sequence increases CD70 expression in lupus T cells and T cells treated with lupusinducing drugs. J Immunol 2005; 174:6212e9.
Strickland F M, Hewagama A, Lu Q, Wu A, Hinderer R, Webb R, et al. Environmental exposure, estrogen and two X chromosomes are required for disease development in an epigenetic model of lupus. J Autoimmun 2012; 38: J135e43.
Pisitkun P, Deane J A, Difilippantonio M J, Tarasenko T, Satterthwaite A B, Bolland S. Autoreactive B cell responses to RNA-related antigens due to TLR7 gene duplication. Science 2006; 312:1669e72.
Chagnon P, Schneider R, Hebert J, Fortin P R, Provost S, Belisle C, et al. Identification and characterization of an Xp22.33;Yp11.2 translocation causing a triplication of several genes of the pseudoautosomal region 1 in an XX male patient with severe systemic lupus erythematosus. Arthritis Rheum 2006; 54:1270e8.
Liu Y, Kuick R, Hanash S, Richardson B. DNA methylation inhibition increases T cell KIR expression through effects on both promoter methylation and transcription factors. Clin Immunol 2009; 130:213e24.
Higuchi T, Aiba Y, Nomura T, Matsuda J, Mochida K, Suzuki M, et al. Cutting edge: ectopic expression of CD40 ligand on B cells induces lupus-like autoimmune disease. J Immunol 2002; 168:9e12.
Peng S L. Altered T and B lymphocyte signaling pathways in lupus. Autoimmun Rev 2009; 8:179e83.
Love D C, Hanover J A. The hexosamine signaling pathway: deciphering the “OGlcNAc code”. Sci STKE 2005; 2005:re13.
Hart G W, Housley M P, Slawson C. Cycling of O-linked beta-Nacetylglucosamine on nucleocytoplasmic proteins. Nature 2007; 446:1017e22.
Zachara N E, Hart G W. Cell signaling, the essential role of O-GlcNAc! Biochim Biophys Acta 2006; 1761:599e617.
Shafi R, Iyer S P, Ellies LG, O'Donnell N, Marek K W, Chui D, et al. The O-GlcNAc transferase gene resides on the X chromosome and is essential for embryonic stem cell viability and mouse ontogeny. Proc Natl Acad Sci USA 2000; 97: 5735e9.
O'Donnell N, Zachara N E, Hart G W, Marth J D. Ogt-dependent X-chromosomelinked protein glycosylation is a requisite modification in somatic cell function and embryo viability. Mol Cell Biol 2004; 24:1680e90.
Kearse K P, Hart G W. Topology of O-linked N-acetylglucosamine in murine lymphocytes. Arch Biochem Biophys 1991; 290:543e8.
Tsokos G C, Nambiar M P, Juang Y T. Activation of the Ets transcription factor Elf-1 requires phosphorylation and glycosylation: defective expression of activated Elf-1 is involved in the decreased TCR zeta chain gene expression in patients with systemic lupus erythematosus. Ann N Y Acad Sci 2003; 987:240e5.
Huang J B, Clark A J, Petty H R. The hexosamine biosynthesis pathway negatively regulates IL-2 production by Jurkat T cells. Cell Immunol 2007; 245:1e6.
Yao A Y, Tang H Y, Wang Y, Feng M F, Zhou R L. Inhibition of the activating signals in NK92 cells by recombinant GST-sHLA-Gla chain. Cell Res 2004; 14:155e60.
Naramura M, Kole H K, Hu R J, Gu H. Altered thymic positive selection and intracellular signals in Cbl-deficient mice. Proc Natl Acad Sci USA 1998; 95:15547e52.
Brembilla N C, Weber J, Rimoldi D, Pradervand S, Schutz F, Pantaleo G, et al. c-Cbl expression levels regulate the functional responses of human central and effector memory CD4 T cells. Blood 2008; 112:652e60.
Methi T, Berge T, Torgersen K M, Tasken K. Reduced Cbl phosphorylation and degradation of the zeta-chain of the T-cell receptor/CD3 complex in T cells with low Lck levels. Eur J Immunol 2008; 38:2557e63.
Wang H Y, Altman Y, Fang D, Elly C, Dai Y, Shao Y, et al. Cbl promotes ubiquitination of the T cell receptor zeta through an adaptor function of Zap-70. J Biol Chem 2001; 276:26004e11.
Balagopalan L, Barr V A, Sommers C L, Barda-Saad M, Goyal A, Isakowitz M S, et al. c-Cbl-mediated regulation of LAT-nucleated signaling complexes. Mol Cell Biol 2007; 27:8622e36.
Fullwood M J, Liu M H, Pan Y F, Liu J, Xu H, Mohamed Y B, et al. An oestrogenreceptor-alpha-boundhuman chromatin interactome. Nature 2009; 462:58e64.
Kotzin, B. L., Systemic lupus erythematosus. Cell, 1996. 85(3): p. 303-6.
MacLeod, A. R., J. Rouleau, and M. Szyf, Regulation of DNA methylation by the Ras signaling pathway. J Biol Chem, 1995. 270(19): p. 11327-37.
Rouleau, J., A. R. MacLeod, and M. Szyf, Regulation of the DNA methyltransferase by the Ras-AP-1 signaling pathway. J Biol Chem, 1995. 270(4): p. 1595-601.
Cornacchia, E., et al., Hydralazine and procainamide inhibit T cell DNA methylation and induce autoreactivity. J Immunol, 1988. 140(7): p. 2197-200.
Lu, Q., et al., Demethylation of ITGAL (CD11a) regulatory sequences in systemic lupus erythematosus. Arthritis Rheum, 2002. 46(5): p. 1282-91.
Richardson, B., et al., Lymphocyte function-associated antigen 1 overexpression and T cell autoreactivity. Arthritis Rheum, 1994. 37(9): p. 1363-72.
Kaplan, M. J., et al., Demethylation of promoter regulatory elements contributes to perforin overexpression in CD4+ lupus T cells. J Immunol, 2004. 172(6): p. 3652-61.
Oelke, K., et al., Overexpression of CD70 and overstimulation of IgG synthesis by lupus T cells and T cells treated with DNA methylation inhibitors. Arthritis Rheum, 2004. 50(6): p. 1850-60.
Basu, D., et al., Stimulatory and inhibitory killer Ig-like receptor molecules are expressed and functional on lupus T cells. J Immunol, 2009. 183(5): p. 3481-7.
Deng, C., et al., Hydralazine may induce autoimmunity by inhibiting extracellular signal-regulated kinase pathway signaling. Arthritis Rheum, 2003. 48(3): p. 746-56.
Deng, C., et al., Decreased Ras-mitogen-activated protein kinase signaling may cause DNA hypomethylation in T lymphocytes from lupus patients. Arthritis Rheum, 2001. 44(2): p. 397-407.
Lu, Q., A. Wu, and B. C. Richardson, Demethylation of the Same Promoter Sequence Increases CD70 Expression in Lupus T Cells and T Cells Treated with Lupus-Inducing Drugs. J Immunol, 2005. 174(10): p. 6212-6219.
Lu, Q., et al., DNA methylation and chromatin structure regulate T cell perforin gene expression. J Immunol, 2003. 170(10): p. 5124-32.
Sawalha, A. H., et al., Defective T-cell ERK signaling induces interferon-regulated gene expression and overexpression of methylation-sensitive genes similar to lupus patients. Genes Immun, 2008. 9(4): p. 368-78.
Gorelik, G., et al., Impaired T cell protein kinase C delta activation decreases ERK pathway signaling in idiopathic and hydralazine-induced lupus. J Immunol, 2007. 179(8): p. 5553-63.
Nishizuka, Y., Protein kinase C and lipid signaling for sustained cellular responses. FASEB J, 1995. 9(7): p. 484-96.
Oates, J. C., The biology of reactive intermediates in systemic lupus erythematosus. Autoimmunity, 2010. 43(1): p. 56-63.
Wang, G., et al., Markers of oxidative and nitrosative stress in systemic lupus erythematosus: Correlation with disease activity. Arthritis Rheum, 2010. 62(7): p. 2064-2072.
Li, L., et al., Protein kinase Cdelta targets mitochondria, alters mitochondrial membrane potential, and induces apoptosis in normal and neoplastic keratinocytes when overexpressed by an adenoviral vector. Mol Cell Biol, 1999. 19(12): p. 8547-58.
Santiago-Raber, M. L., et al., Genetic basis of murine lupus. Autoimmun Rev, 2004. 3(1): p. 33-9.
Gschwendt, M., Protein kinase C delta. Eur J Biochem, 1999. 259(3): p. 555-64.
Mecklenbrauker, I., et al., Protein kinase Cdelta controls self-antigen-induced B-cell tolerance. Nature, 2002. 416(6883): p. 860-5.
Miyamoto, A., et al., Increased proliferation of B cells and auto-immunity in mice lacking protein kinase Cdelta. Nature, 2002. 416(6883): p. 865-9.
Gruber, T., et al., PKCdelta is involved in signal attenuation in CD3+ T cells. Immunol Lett, 2005. 96(2): p. 291-3.
Richardson, B., Effect of an inhibitor of DNA methylation on T cells. II. 5-Azacytidine induces self-reactivity in antigen-specific T4+ cells. Hum Immunol, 1986. 17(4): p. 456-70.
Oates, J. C. and G. S. Gilkeson, The biology of nitric oxide and other reactive intermediates in systemic lupus erythematosus. Clinical Immunology, 2006. 121(3): p. 243-250.
Nath, S. K., J. Kilpatrick, and J. B. Harley, Genetics of human systemic lupus erythematosus: the emerging picture. Curr Opin Immunol, 2004. 16(6): p. 794-800.

We claim:

1. A method of diagnosing lupus, assessing a subject's risk for developing lupus, and/or determining lupus disease activity in a subject comprising detecting one or more epigenetic markers of lupus.

2. The method of claim 1, wherein detecting one or more epigenetic markers of lupus comprise oxidation-related modifications of PKCδ in the subject.

3. The method of claim 2, wherein the oxidation-related modifications comprise nitration of PKCδ.

4. The method of claim 2, wherein detecting oxidation-related modifications of PKCδ comprises determining the level of oxidation-related modifications of PKCδ in the subject.

5. The method of claim 3, wherein detecting oxidation-related modifications of PKCδ further comprises comparing the level of oxidation-related modifications of PKCδ to a control or threshold level that is indicative of lupus, a risk of developing lupus, and/or active lupus in the subject.

6. The method of claim 1, wherein the one or more epigenetic markers of lupus comprises X-chromosome demethylation.

7. The method of claim 1, wherein the one or more epigenetic markers of lupus comprises overexpression of one or more of CD40LG, CXCR3, OUT, miR-98, let-7f-2, miR 188 3p, miR-421, and miR-503.

8. The method of claim 1, wherein detecting one or more epigenetic markers of lupus comprises in vitro analysis.

9. The method of claim 1, wherein detecting one or more epigenetic markers of lupus comprises antibody detection.

10. A method of monitoring treatment of lupus comprising:
(a) detecting one or more epigenetic markers of lupus in a subject;
(b) administering a treatment for lupus to the subject;
(c) repeating the detection of one or more epigenetic markers of lupus in the subject;
(d) comparing the epigenetic markers detected in steps (a) and (c), wherein a reduction in one or more of the epigenetic markers of lupus indicates benefit of the treatment.

11. The method of claim 10, wherein treating comprises reducing the oxidative stress in the subject.

12. The method of claim 10, wherein treating comprises administering a lupus therapeutic.

13. The method of claim 10, wherein said one or more epigenetic markers of lupus comprises oxidation-related modifications of PKCδ in the subject.

14. The method of claim 13, wherein the oxidation-related modifications comprise nitration of PKCδ.

15. The method of claim 13, wherein the reduction in one or more of the epigenetic markers of lupus a reduction in oxidation-related modifications of PKCδ.

16. The method of claim 10, wherein the one or more epigenetic markers of lupus comprises X-chromosome demethylation.

17. The method of claim 10, wherein the one or more epigenetic markers of lupus comprises overexpression of one or more of CD40LG, CXCR3, OUT, miR-98; let-7f-2, miR 188 3p, miR-421, and miR-503.

18. A non-human transgenic mammal exhibiting decreased expression or activity of PKCδ.

19. The non-human transgenic mammal of claim 18, wherein decreased PKCδ expression or activity, when present, is limited to CD4+ cells.

20. The non-human transgenic mammal of claim 18, wherein PKCδ inactivation is only expressed in the presence of an inducer.

21. The non-human transgenic mammal of claim 20, wherein the inducer comprises Doxycycline.


SEQUENCE LISTING (18 sequences)

SEQ ID NO: 1 - DNA, 22 bp, Artificial Sequence
  Synthetic
caacgaccccttcattgacctc

SEQ ID NO: 2 - DNA, 24 bp, Artificial Sequence
  Synthetic
gcctcaccccatttgatgttagtg

SEQ ID NO: 3 - DNA, 19 bp, Artificial Sequence
  Synthetic
tggctgtgggcatctgctc

SEQ ID NO: 4 - DNA, 22 bp, Artificial Sequence
  Synthetic
acatctccgtggaccaggtatg

SEQ ID NO: 5 - DNA, 25 bp, Artificial Sequence
  Synthetic
ggaaggctacctggctaaagtcaag

SEQ ID NO: 6 - DNA, 24 bp, Artificial Sequence
  Synthetic
actgaaagggtgtcactgtccgac

SEQ ID NO: 7 - DNA, 21 bp, Artificial Sequence
  Synthetic
ttcagtgatagagaacgtatg

SEQ ID NO: 8 - DNA, 25 bp, Artificial Sequence
  Synthetic
cagcacagaaaggctggcttgcttc

SEQ ID NO: 9 - RNA, 23 nt, Homo sapiens
aagcagaauauucuccuaccuca

SEQ ID NO: 10 - RNA, 22 nt, Homo sapiens
ugagguaguaaguuguauuguu

SEQ ID NO: 11 - RNA, 23 nt, Homo sapiens
cucccuccucucuaccuaccucu

SEQ ID NO: 12 - RNA, 22 nt, Homo sapiens
ugagguaguaaguuguauuguu

SEQ ID NO: 13 - RNA, 23 nt, Homo sapiens
aagucuugcccucucugugggau

SEQ ID NO: 14 - RNA, 21 nt, Homo sapiens
cucccacaugcaggguuugca

SEQ ID NO: 15 - RNA, 23 nt, Homo sapiens
uacuggggcccaagaugugggau

SEQ ID NO: 16 - RNA, 21 nt, Homo sapiens
cucccacaugcaggguuugca

SEQ ID NO: 17 - RNA, 23 nt, Homo sapiens
ucuggguugguuccagugggaaa

SEQ ID NO: 18 - RNA, 21 nt, Homo sapiens
cucccacaugcaggguuugca